(19) United States
(12) Patent Application Publication
(10) Pub. No.: US 2026/0328771 A1
(43) Pub. Date: Oct. 22, 2026

(54) METHODS FOR ASSESSING COLORECTAL CANCER USING EXTRACELLULAR VESICLES

(71) Applicant: MESO SCALE TECHNOLOGIES, LLC., Rockville, MD (US)

(72) Inventors: David ROUTENBERG, Gaithersburg, MD (US); Lucie HEBERT, Bethesda, MD (US); George SIGAL, Rockville, MD (US)

(21) Appl. No.: 19/486,522

(22) PCT Filed: May 1, 2024
(86) PCT No.: PCT/US2024/027283

§ 371 Date: Nov. 21, 2025

Related U.S. Application Data
(60) Provisional application No. 63/504,049, filed on May 24, 2023.

(51) Int. Cl. G01N 33/57535; G01N 33/575

(52) U.S. Cl. CPC G01N 33/57535; G01N 33/57565

(57) ABSTRACT

The present disclosure provides methods for detecting, diagnosing, monitoring response to treatment or recurrence, or predicting overall survival, progression-free survival or duration of response in a subject suspected of having colorectal cancer (CRC) comprising performing one or more assays on at least one biological sample from the subject to detect one or more specific populations of EVs.

### STATEMENT REGARDING FEDERALLY SPONSORED RESEARCH AND DEVELOPMENT

This invention was made with government support under grant number TR002886 awarded by the National Institutes of Health. The government has certain rights in the invention.

### FIELD OF THE INVENTION

This invention relates to methods for detecting colorectal cancer (CRC)-derived extracellular vesicles in a sample and for detecting CRC in a subject. Methods for aiding in the detection of CRC, staging CRC, determining a prognosis in a CRC subject, and monitoring CRC in a subject are additionally disclosed.

### BACKGROUND

Extracellular vesicles (EVs) are a diverse class of lipid bilayer-enclosed particles that are released by all cell types under both normal and pathological conditions and are detected in all tissues and biofluids. The most well-characterized EV classes are exosomes, small EVs (40 nm-150 nm diameter) derived from the late-endosome, and microvesicles, EVs which are shed from the plasma membrane and exhibit a broader size distribution (40 nm-1 μm diameter). Once released into the extracellular space, these populations are likely indistinguishable as their molecular compositions are similar and their size ranges overlap. Often the functional term small extracellular vesicle (sEV) is used to encompass the mixture of both populations that fall under ~200 nm. Other populations of larger vesicles are produced by cells under specific conditions, including apoptotic bodies, typically >500 nm, and large oncosomes (>1 μm) which are shed in rare cases from tumor cells (>1 μm). Recently several groups have reported the existence of new classes of amembranous, non-vesicular nanoparticles (NVNPs) such as exomeres and supermeres (30-40 nm and 15-25 nm, respectively). It is now accepted that EVs have numerous roles in inter-cellular communication, facilitated by the transfer of EV cargo to recipient cells following uptake, or by the interaction of EV surface proteins with cellular receptors. EV secretion is involved in the maintenance of normal physiological functions and is also linked to numerous disease states, including certain cancers, cardiovascular disease, neurological and immunological disorders. Methods for detecting and isolating EVs are described, for example, in international patent application publication numbers WO2019222708, WO2020086751, and WO2022051481.

Colorectal cancer (CRC) is the third most common cancer worldwide, accounting for almost 10% of all cases, and is the second leading cause of cancer death in the United States. CRC recurrence rate averages 25%, and follow-up after curative treatment remains a complex challenge for the healthcare system, in terms of diagnostics, treatment and monetary costs.

The tumor-node-metastasis (TNM) classification and staging system is used to assign a patient's CRC stage according to the characteristics of the primary tumor (T) and the extent of regional lymph node involvement (N) and distant metastasis (M). In general, the survival of patients with advanced-stage cancers is lower than that of patients with early-stage cancers, although some survival paradox has been reported between stage IIB/C and stage IIIA. Other prognostic markers are used as indicators of the appropriate therapeutic strategy, but are excluded from the classification, including the histological grade, RAS/BRAF mutation, and plasma carcinoembryonic antigen (CEA) levels.

CEA, also known as CEACAM5 or CD66e, is a heavily glycosylated protein that belongs to the CEA-related cell adhesion molecule (CEACAM) family of the immunoglobulin (Ig) gene superfamily. CEA has a luminal expression pattern in some normal epithelial tissues including colon, stomach and cervix. CEA in normal tissues protects the luminal cell membranes from microbial infection by binding and trapping infectious microorganisms. In contrast, its expression is frequently high in various carcinomas including the gastrointestinal and digestive tract, lung and breast cancers. CEA in some cancer cells not only loses its polarized expression, but is also shed from the cancer cells plasma membrane by active cleavage of its GPI-anchor, resulting in elevated plasma concentrations. Elevated CEA levels in both tumors and in plasma of some cancer patients makes it an attractive therapeutic target and prognostic marker.

Plasma CEA concentration is assessed routinely during the postoperative follow-up of CRC patients (after tumor removal), and it is also used for prognostic evaluation given its superiority to other standalone prognostic biomarkers. However, the results from measuring CEA alone in patient plasma using current methods have a low predictive value for diagnosis in asymptomatic CRC patients with a relatively low sensitivity of 46% (95% confidence interval (CI) 0.45 to 0.47) and specificity 89% (95% CI 0.88 to 0.92). While the lack of specificity is partly explained by the fact that CEA can be elevated due to both malignant and benign conditions, the lack of sensitivity has yet to be explained. There remains a need for improved methods for detecting levels of CEA and other biomarkers in patients diagnosed with, or suspected of having, CRC wherein the resulting detected levels are indicative of the patient's CRC status, diagnosis, stage, and/or prognosis.

### SUMMARY OF THE INVENTION

Aspects of the present disclosure provide methods for detecting colorectal tumor-derived EVs in a sample from a subject suspected of having a colorectal tumor or suspected of having colorectal cancer (CRC), methods for diagnosing CRC or the presence of a colorectal tumor in a subject suspected of having CRC or suspected of having a colorectal tumor, methods for staging CRC in a subject, methods for determining a prognosis of a subject diagnosed with CRC or suspected of having CRC, methods for aiding in the detection of colorectal cancer (CRC) or a colorectal tumor in a subject suspected of having CRC or a colorectal tumor, methods for aiding in the staging of CRC in a subject, methods for assisting in determining a prognosis of a subject diagnosed with CRC or suspected of having CRC, methods for monitoring CRC recurrence in a subject previously classified as being in CRC remission, and methods for monitoring response to CRC treatment in a subject receiving CRC treatment.

In aspects, the present disclosure provides method for detecting, diagnosing, staging, monitoring changes or recurrence, or predicting response of a colorectal tumor to a treatment in a subject comprising: (a) performing an assay on a biological sample from the subject to detect a level of at least one specific population of EVs in the biological sample; (b) comparing the level of specific EVs to a reference level; and (c) determining the presence, diagnosis, stage, progression, or response of the tumor based on the level of EVs in one or more samples relative to the reference.

In aspects, the present disclosure provides methods for detecting, diagnosing, staging, monitoring changes or recurrence, or predicting response of a colorectal tumor to a treatment in a subject comprising: (a) performing an assay on a biological sample from the subject to detect a level of CEA+EVs or CD73+EVs, or a combination thereof; (b) comparing the level of CEA+EVs CD73+EVs or a combination thereof to a reference level; and (c) determining the presence, diagnosis, stage, progression, or response of the tumor based on the level of EVs in one or more samples relative to the reference.

In aspects, the methods disclosed herein comprise performing an assay on a biological sample from a subject to detect extracellular vesicles comprising carcinoembryonic antigen (CEA+EVs) in the biological sample, performing an assay on the biological sample from the subject to detect soluble CEA in the biological sample, and determining whether the combined level of soluble CEA and CEA+EVs in the biological sample from the subject exceeds the combined level of CEA+EVs and soluble CEA in a reference. In embodiments, the methods further comprise:
diagnosing the subject as having CRC if the combined level of soluble CEA and CEA+EVs in the biological sample from the subject exceeds the combined level of CEA+EVs and soluble CEA in the reference; or
classifying the subject as having stage IV CRC if the combined level of soluble CEA and CEA+EVs in the biological sample from the subject exceeds the combined level of CEA+EVs and soluble CEA in the reference; or
determining that the subject has a poor prognosis if the combined level of soluble CEA and CEA+EVs in the biological sample from the subject exceeds the combined level of CEA+EVs and soluble CEA in the reference.

In embodiments, a combined level of CEA+EVs and soluble CEA in the biological sample that is greater than the combined level of CEA+EVs and soluble CEA in the reference aids in detecting CRC in the subject.

In embodiments, a combined level of CEA+EVs and soluble CEA in the biological sample that is greater than the combined level of CEA+EVs and soluble CEA in the reference aids in classifying the subject as having stage IV CRC.

In embodiments, a combined level of CEA+EVs and soluble CEA in the biological sample that is greater than the combined level of CEA+EVs and soluble CEA in the reference aids in determining that the subject has a poor prognosis.

In embodiments, wherein a combined level of CEA+EVs and soluble CEA in the biological sample that is greater than the combined level of CEA+EVs and soluble CEA in the reference aids in determining that CRC has recurred in the subject.

In embodiments of the methods disclosed herein, the method further comprises communicating the method results to a healthcare provider. In embodiments of the methods disclosed herein, the method further comprises administering a CRC treatment regimen to the subject.

Aspects of the methods disclosed herein comprise performing an assay on a first biological sample obtained from a subject before CRC treatment administration to detect CEA+EVs in the first biological sample, performing an assay on said first biological sample obtained from the subject before CRC treatment administration to detect soluble CEA in the first biological sample, performing an assay on a second biological sample obtained from the subject after CRC treatment administration to detect CEA+EVs in said second biological sample, and performing an assay on said second biological sample obtained from the subject after CRC treatment administration to detect soluble CEA in said second biological sample, wherein a combined level of CEA+EVs and soluble CEA in the second biological sample that is greater than or equal to the combined level of CEA+EVs and soluble CEA in the first biological sample indicates that the subject is not responding favorably to said treatment, and wherein the second biological sample is obtained from the subject at least 7 days to at least 6 months after administration of said CRC treatment to said subject.

In embodiments of the disclosed methods, the assay to detect CEA+EVs comprises treating the biological sample to remove soluble proteins and other interfering substances from the sample to produce a purified product prior to detecting CEA+EVs. The present disclosure further provides a method for detecting colorectal tumor-derived EVs in a sample from a subject suspected of having a colorectal tumor or suspected of having CRC, the method comprising detecting EVs that comprise both CEA and CD73 (CEA+/CD73+EVs) in the sample.

Samples employed in the methods disclosed herein may be, for example, biological samples such as plasma, whole blood, serum, or a liquid biopsy.

### BRIEF DESCRIPTION OF THE DRAWINGS

The following drawings form part of the present specification and are included to further demonstrate exemplary embodiments of certain aspects of the present invention.

FIG. 1: Soluble CEA and EV-associated CEA are elevated in distinct subsets of stage IV CRC patients. (A) Soluble CEA was estimated from ECL signals using CEA recombinant protein (Sino Biologicals) of known concentrations. Concentrations (ng/mL) are displayed on a log scale. Compared to the normal group, soluble CEA level was higher in stage II (p=0.008), in stage III (p=0.001), and in stage IV (p<0.0001). (B) EV-associated CEA was measured by assessing intact EVs expressing CEA and EV-specific tetraspanins (CD81, CD63 and CD9) and ECL signal is displayed on a log-scale. EV-associated CEA level was comparable between normal samples and stages I to III patients, but was markedly elevated in stage IV (p=0.0004). The dashed line indicates 3-fold of the ECL signal from EVs captured with an isotype control (IgG) and is indicative of non-specific signal. (C) Soluble CEA and CEA+EVs were plotted in a two-dimensional graph on a log scale. For both axis, median values of the normal group+3 StDev was used to make the quadrants. Normal samples cluster in the bottom left quadrant, stage IV CRC patients are dispersed across the quadrants, suggesting three distinct patterns of CEA expression. The upper left quadrant reveals a subset a patients (N=5) with high EV-associated CEA and low level of soluble CEA, the upper right quadrant shows patients (N=6) with high levels of both soluble and EV-associated CEA, and the bottom right quadrant shows patients with elevated soluble CEA only (N=8). (D) Receiver operating characteristic (ROC) curves were plotted for each assay alone or in combination, and the area under the curves (AUCs) are indicated in the legend. The standard soluble CEA assay and the EV-associated CEA assay were 0.927 and 0.803, respectively, and AUC was 0.967 when the two assays were combined. Mann-Whitney 2-tailed non-parametric test was used to test the significance of CEA levels between the groups. (E) EV-associated CD73 was measured by assessing intact EVs expressing CD73 and EV-specific tetraspanins (CD81, CD63 and CD9) and ECL signal is displayed on a log-scale. EV-associated CD73 level was comparable between normal samples and stages I to III patients, but was markedly elevated in stage IV (p=0.0024). The dashed line indicates 3-fold of the ECL signal from EVs captured with an isotype control (IgG) and is indicative of non-specific signal.

FIG. 2: Detecting CEA in co-localization with CD73 on EVs improves specificity of detecting CRC derived EVs. (A) Intact EVs displaying both CEA and CD73 were assessed using a combination of antibodies targeting CEA, CD73 and tetraspanins, and ECL signal is displayed on a log-scale. CEA+/CD73+EV level was comparable between normal samples and stages I to III patients, but was markedly elevated in stage IV (p=0.0074). The dashed line indicates 3-fold of the ECL signal from EVs captured with an isotype control (IgG) and is indicative of non-specific signal. (B) ECL signals were normalized by geometric mean of the entire cohort. Normalized ECL signal from EVs detected either with CEA and tetraspanins or CEA, CD73 and tetraspanins was plotted against each other on a log scale. The slope of the linear fit indicates 3-fold improvement of signal to background for the CEA+/CD73+EV assay relative to the CEA+EV assay. (C) Receiver operating characteristic (ROC) curves were plotted for each assay alone or in combination, and the area under the curves (AUCs) are indicated in the legend. The combination of soluble CEA and CEA+/CD73+EVs shows the highest AUC of 0.975. Mann-Whitney 2-tailed non-parametric test was used to test the significance of CEA levels between the groups.

FIG. 3A-3F are Kaplan-Meier curves showing Overall Survival in the CaboMab cohort at the First Timepoint for A) sCD73, B) sCEA, C) EV_CEA, D) EV_CD73, E) EV_CD73CD73, and F) EV_CEACD73.

FIG. 4A-4F are Kaplan-Meier curves showing Overall Survival in the CaboMab cohort at the Second Timepoint for A) sCD73, B) sCEA, C) EV_CEA, D) EV_CD73, E) EV_CD73CD73, and F) EV_CEACD73.

FIG. 5A-D are Kaplan-Meier curves showing Overall Survival in the X-TRAP cohort at the First Timepoint for A) sCD73, B) EV_CEA, C) EV_CD73CD73, and D) EV_CEACD73.

FIG. 6A-D are plots of Kaplan-Meier curves showing Overall Survival in the X-TRAP cohort at the Second Timepoint for A) sCD73, B) EV_CEA, C) EV_CD73CD73, and D) EV_CEACD73.

FIG. 7A-B are Kaplan-Meier curves showing Overall Survival in the CaboMab cohort for sCEA or EV_CEA at A) the First Timepoint, and B) the Second Timepoint. High bin includes all patients that have sCEA above median OR EV_CEA above median.

FIG. 8A-8D are Kaplan-Meier curves showing Progression-Free Survival in the CaboMab cohort at the First Timepoint for A) sCD73, B) EV_CEA, C) EV_CD73CD73, and D) EV CEACD73.

FIG. 9A-9D are Kaplan-Meier curves showing Progression-Free Survival in the CaboMab cohort at the Second Timepoint for A) sCD73, B) EV_CEA, C) EV_CD73CD73, and D) EV_CEACD73.

### DETAILED DESCRIPTION

Any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” The use of the term/phrase “and/or” when used with a list means one or more of the listed items may be utilized, e.g., it is not limited to one or all of the elements.

The term “CEA+EVs” as used herein refers to extracellular vesicles (“EVs”) that present the surface marker CEA (also referred to as CEACAM5).

The term “CD73+EVs” as used herein refers to extracellular vesicles that present the surface marker CD73.

The term “CEA+CD73+” as used herein refers to extracellular vesicles that present both the surface marker CEA and the surface marker CD73.

The term “tetraspanin” (also referred to as TTSP) as used herein refers to surface markers that are associated with EVs. This term includes, but is not limited to, CD9, CD37, CD63, CD81, and CD82.

### I. Overview

Extracellular vesicles (EVs) include a broad spectrum of vesicles secreted by several types of cells and the term is used as a collective one. These include exosomes, ectosomes, oncosomes, shed vesicles, microvesicles, and apoptotic bodies. Thus, EVs represent a broad spectrum of vesicles secreted by several types of cells. Major groups include exosomes (endosomal origin, 40-200 nm), microvesicles/ectosomes (plasma membrane origin, 100-1000 nm) and larger particles such as large-oncosomes (tumor cell origin, >1 μm). The exact definition and nomenclature for each of these general vesicles classes has yet to be fully codified by the field due to their heterogeneous nature, herein, the term “EVs” is as defined by the International Society of Extracellular Vesicles (see Gardiner et al., Journal of Extracellular Vesicles 5 (1): 32945 (2016).

EVs released from a variety of cells target recipient cells for intercellular communication and transfer a subset of genetic materials, proteins, lipids, and metabolites. EVs may comprise one or more proteins on or embedded in their surface or in their lumen (i.e., as cargo). An EV may comprise a protein that is covalently or non-covalently bound to one or more surface markers and/or structural components of the EV surface. An EV may comprise a protein that is associated with the EV membrane. An EV may comprise a protein that is associated with EV transmembrane protein(s).

In embodiments, he present disclosure provides methods for detecting, diagnosing, staging, monitoring changes or recurrence, or predicting response of a colorectal tumor to a treatment in a subject comprising performing an assay on a biological sample from the subject to detect a level of at least one specific population of EVs in the biological sample, comparing the level of specific EVs to a reference level, and determining the presence, diagnosis, stage, progression, or response of the tumor based on the level of EVs in one or more samples relative to the reference. In some embodiments, the specific population of EVs is defined by the presence of at least one surface marker selected from CEA (CEACAM5), CD73 or a combination thereof.

In some embodiments, assays are performed on at least one biological sample from the subject to detect a soluble biomarker. In some embodiments, the soluble biomarker is CEA, CD73, or a combination thereof. In some embodiments, the surface marker defining the EV population and the soluble biomarker are substantially the same protein. In some embodiments, the reference level is determined from a biological sample from a second subject without a colorectal tumor, from a biological sample from the same subject, or from a synthetic sample. In some embodiments, the synthetic sample is a calibrator. In some embodiments, the monitoring changes comprises determining the colorectal tumor size or stage between two timepoints. In some embodiments, the monitoring changes comprises determining overall survival, progression free survival, response to treatment, or duration of response to treatment.

In some embodiments, the specific population of EVs is identified by the presence of CEA (CEACAM5), and wherein the method comprises predicting overall survival, progression-free survival, duration of response to treatment, or a combination thereof. In some embodiments, performing an assay comprises performing a 3-marker proximity assay comprising a first detection reagent that binds CEA+EVs or CD73+EVs, and a second and third binding reagent that binds CEA+EVs, CD73EVs, or a tetraspanin, wherein the first binding reagent is a capture reagent and the second and third binding reagents are detection reagents. In some embodiments, the tetraspanin is CD63, CD81, and/or CD9.

In embodiments, the present disclosure provides a method for monitoring response to CRC treatment in a subject receiving CRC treatment, the method comprising: performing an assay on a first biological sample obtained from the subject before CRC treatment administration to detect CEA+CD73+EVs or CD73+EVs in the first biological sample; and performing an assay on a second biological sample obtained from the subject after CRC treatment administration to detect CEA+CD73+EVs or CD73+EVs in the second biological sample; wherein a level of CEA+CD73+Evs or CD73+EVs in the second biological sample that is greater than or equal to a level of CEA+CD73+EVs or CD73+EVs in the first biological sample indicates that the subject is not responding favorably to said treatment; and wherein the second biological sample is obtained from the subject 7 days to 6 months after administration of the CRC treatment to the subject.

The present disclosure provides methods comprising performing an assay on a biological sample from a subject to detect EVs comprising carcinoembryonic antigen (CEA+EVs) in the biological sample, performing an assay on the biological sample from the subject to detect soluble CEA in the biological sample, and comparing the combined level of soluble CEA and CEA+EVs in the biological sample with the combined level of soluble CEA and CEA+EVs in a reference. The present inventors surprisingly discovered that the level of CEA+EVs in subjects with colorectal cancer provides distinct information from that ascertained from the level of soluble CEA, alone, in the subjects, and that assays combining these two metrics lead to improved performance for CRC detection or assessment relative to the gold-standard soluble CEA assay. A combined level of CEA+EVs and soluble CEA in the biological sample that is greater than the combined level of CEA+EVs and soluble CEA in a reference (e.g., combined levels in a non-CRC population) is useful for determining a variety of indications. The present inventors further unexpectedly discovered that testing for CD73 and CEA double positive EVs (CEA+/CD73+EVs) further improves discrimination of subjects with colorectal cancer and normal subjects.

In one aspect of the invention, the above method is used for diagnosing CRC or the presence of a colorectal tumor in a subject, wherein when a combined level of CEA+EVs and soluble CEA in the biological sample is greater than the combined level of CEA+EVs and soluble CEA in the reference, the subject is diagnosed as having CRC or a colorectal tumor. In embodiments, the subject is suspected of having CRC or of having a colorectal tumor. In embodiments, the subject is diagnosed with stage IV CRC. In embodiments, the CEA+EVs comprise both CEA and CD73 (CEA+/CD73+EVs).

In another aspect of the invention, the above method is used for aiding in the detection of CRC or a colorectal tumor in a subject, wherein a combined level of CEA+EVs and soluble CEA in the biological sample that is greater than the combined level of CEA+EVs and soluble CEA in the reference aids in detecting CRC in the subject. In embodiments, the subject is suspected of having CRC, stage IV CRC, or a colorectal tumor. In embodiments, the method aids in the detection of stage IV CRC in the subject. In embodiments, the CEA+EVs comprise both CEA and CD73 (CEA+/CD73+EVs).

In another aspect of the invention, the above method is used for staging CRC in a subject, wherein when a combined level of CEA+EVs and soluble CEA in the biological sample is greater than the combined level of CEA+EVs and soluble CEA in the reference, the subject is classified as having stage IV CRC. In embodiments, the subject is suspected of having or has been diagnosed with CRC, stage IV CRC, or a colorectal tumor. In embodiments, the CEA+EVs comprise both CEA and CD73 (CEA+/CD73+EVs).

In another aspect of the invention, the above method is used for aiding in the staging of CRC in a subject, wherein a combined level of CEA+EVs and soluble CEA in the biological sample that is greater than the combined level of CEA+EVs and soluble CEA in the reference aids in classifying the subject as having stage IV CRC. In embodiments, the subject is suspected of having, or has been diagnosed with, CRC, stage IV CRC, or a colorectal tumor. In embodiments, the CEA+EVs comprise both CEA and CD73 (CEA+/CD73+EVs).

In another aspect of the invention, the above method is used for determining a prognosis of a subject diagnosed with CRC or suspected of having CRC, wherein when a combined level of CEA+EVs and soluble CEA in the biological sample is greater than the combined level of CEA+EVs and soluble CEA in the reference, the subject is determined to have a poor prognosis. In embodiments, the subject is suspected of having late-stage CRC or stage IV CRC. In embodiments, the CEA+EVs comprise both CEA and CD73 (CEA+/CD73+EVs). A patient has a poor prognosis if their chances of surviving 5 or more years is less the 5 year survival rate of a healthy person without CRC. In embodiments, a poor prognosis is a<20% likelihood of surviving 5 or more years. In embodiments, a poor prognosis is a<15% likelihood of surviving 5 or more years. In embodiments a poor prognosis is a<10% likelihood of surviving 5 or more years. In embodiments a poor prognosis is a<5% likelihood of surviving 5 or more years.

In another aspect of the invention, the above method is used for assisting in determining a prognosis of a subject diagnosed with CRC or suspected of having CRC, wherein a combined level of CEA+EVs and soluble CEA in the biological sample that is greater than the combined level of CEA+EVs and soluble CEA in the reference aids in determining that the subject has a poor prognosis. In embodiments, the subject is suspected of having late-stage CRC or stage IV CRC. In embodiments, the CEA+EVs comprise both CEA and CD73 (CEA+/CD73+EVs).

In another aspect of the invention, the above method is used for monitoring CRC recurrence in a subject previously classified as being in CRC remission, wherein a combined level of CEA+EVs and soluble CEA in the biological sample that is greater than the combined level of CEA+EVs and soluble CEA in the reference aids in determining that CRC has recurred in the subject. In embodiments, the subject is suspected of having late-stage CRC or stage IV CRC. In embodiments, the CEA+EVs comprise both CEA and CD73 (CEA+/CD73+EVs).

The present disclosure additionally provides a method for monitoring response to CRC treatment in a subject receiving treatment for CRC, the method comprising performing an assay on a first biological sample obtained from the subject before CRC treatment administration to detect CEA+EVs in the first biological sample, performing an assay on said first biological sample obtained from the subject before CRC treatment administration to detect soluble CEA in the first biological sample, performing an assay on a second biological sample obtained from the subject after CRC treatment administration to detect CEA+EVs in said second biological sample, and performing an assay on said second biological sample obtained from the subject after CRC treatment administration to detect soluble CEA in said second biological sample, wherein a combined level of CEA+EVs and soluble CEA in the second biological sample that is greater than or equal to the combined level of CEA+EVs and soluble CEA in the first biological sample indicates that the subject is not responding favorably to said treatment. In some embodiments, the second biological sample is obtained from the subject at least 1, 2, 3, 4, 5, 6, or 7 days after administration of said CRC treatment to said subject. In some embodiments, the second biological sample is obtained from the subject at least 1-4 weeks after administration of said CRC treatment to said subject. In some embodiments, the second biological sample is obtained from the subject at least 1, 2, 3, 4, 5, or 6 months or 1 year after administration of said CRC treatment to said subject. In embodiments, the subject has been diagnosed with late-stage CRC or stage IV CRC. In embodiments, the CEA+EVs comprise both CEA and CD73 (CEA+/CD73+EVs).

The present disclosure additionally provides a method for detecting colorectal tumor-derived EVs in a sample from a subject, the method comprising detecting CEA+/CD73+EVs in the sample. In embodiments, the subject has been diagnosed with, or is suspected of having, a colorectal tumor, CRC, or stage IV CRC.

The methods of the invention can be used alone or in combination with other diagnostic tests or methods to assess patients with, or suspected of having, colorectal cancer.

The detected level of CEA+EVs or the combined levels of CEA+EVs and soluble CEA in a sample may be used to detect or monitor CRC and/or to determine the responsiveness of CRC to a specific treatment regimen. The specific methods/algorithms for using level of CEA+EVs or the combined levels of CEA+EVs and soluble CEA in a sample to make these determinations, as described herein, may optionally be implemented by software running on a computer that accepts the CEA+EV level or combined levels of CEA+EVs and soluble CEA as input and returns a report with the determinations to the user. This software may run on a standalone computer, or it may be integrated into the software/computing system of the analytical device used to measure the CEA+EV level or combined levels of CEA+EVs and soluble CEA or, alternatively, into a laboratory information management system (LIMS) into which crude or processed analytical data is entered.

According to one aspect of the invention, the level of CEA+EVs or the combined levels of CEA+EVs and soluble CEA are measured in samples collected from individuals clinically diagnosed with, suspected of having or at risk of developing CRC. Initial diagnosis may have been carried out using conventional methods. The level of CEA+EVs or the combined levels of CEA+EVs and soluble CEA in a sample are also measured in samples from healthy individuals. The ability for the level of CEA+EVs or the combined levels of CEA+EVs and soluble CEA to distinguish between normal and CRC subjects is identified by visual inspection of the data, for example, by visual classification of data plotted on a one-dimensional or multidimensional graph, or by using statistical methods such as characterizing the statistically weighted difference between healthy, control/non-CRC individuals and CRC subjects and/or by using Receiver Operating Characteristic (ROC) curve analysis. The area under a ROC curve is a measure of the utility of a measured level in the correct identification of one or more diseases or conditions.

The level of CEA+EVs in a reference is the level of CEA+Evs in a normal, non-CRC population with which to compare CEA levels in a biological sample from a subject to determine if the subject has CRC. In embodiments, the combined levels of soluble CEA and CEA+Evs in a reference is a linear combination of the levels of soluble CEA and CEA+Evs in a sample from one or more healthy subjects. In embodiments, the combined levels of soluble CEA and CEA+Evs in a reference is a non-linear combination of the soluble CEA and CEA+Evs levels in a sample from one or more healthy subjects. In embodiments, the combined levels of soluble CEA and CEA+Evs in a reference is a quadratic combination of the soluble CEA and CEA+EVs levels in a sample from one or more healthy subjects. The value of the combined levels of soluble CEA and CEA+EVs in a biological sample for distinguishing between normal and CRC subjects. Patients may be identified by visual inspection of the data, for example, by visual classification of data plotted on a one-dimensional or multidimensional graph, or by using statistical methods such as characterizing the statistically weighted difference between control individuals and CRC subjects and/or by using ROC curve analysis. A variety of suitable methods for setting detection thresholds/algorithms are known in the art and will be apparent to the skilled artisan. In embodiments, the combination of the soluble CEA and CEA EVs levels in a subject is used to produce a single classifier with an improved ROC for discriminating late stage CRC from early stage or healthy controls.

In embodiments of the invention, extracellular vesicles comprising carcinoembryonic antigen (CEA+EVs) comprise CEA on their surface. In embodiments, CEA+EVs comprise CEA in their cargo. In embodiments, CEA+EVs comprise CEA on their surface and in their cargo. A variety of analytical methods have been used to characterize EVs and their encapsulated contents (i.e., cargo) including, most commonly, immunoassays (Western blotting, flow cytometry, sandwich immunoassays), electron microscopy, mass spectrometry, PCR and sequencing, and nanoparticle tracking.

Methods for detecting soluble CEA are known in the art.

The term “antibody” includes for example, monoclonal antibodies, human antibodies, mouse antibodies, rabbit antibodies, humanized antibodies, chimeric antibodies, single-chain Fvs (scFv), disulfide-linked Fvs (sdFv), Fab fragments, F(ab′) fragments, anti-idiotypic (anti-Id) antibodies, and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass.

An “antibody fragment”, as used herein, refers to one or more portions of an antibody that retain the ability to specifically interact with (e.g., by binding, steric hinderance, stabilizing/destabilizing, spatial distribution) an epitope of an analyte of interest. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

I. Assays to Detect CEA+EVs and/or CD73+EVs in a Biological Sample

CEA+EVs and/or CD73+ may be detected in a biological sample using a number of methods including those disclosed in PCT publication numbers WO2019222708, WO2020086751, and WO2022051481, each of which is incorporated herein by reference in its entirety. In embodiments of the methods disclosed herein, a two-marker assay for intact EVs—similar to that disclosed in Example 2.2 of PCT application publication WO2019222708, but using a first binding reagent (capture reagent) that binds CEA or CD73 and a second binding reagent (detect reagent) that binds to a common EV marker, such as CD81, CD63, CD81, or CD9 (detect reagent)—is performed to detect CEA+EVs in a biological sample. In embodiments, the second binding reagent is detectably labeled such that CEA+EVs and/or CD73+EVs in a sample are detected by detecting the detectable label. In embodiments the detectable label is a fluorescent, luminescent, chemiluminescent, or colorimetric label. Detectable labels are discussed further herein.

In embodiments of the methods disclosed herein, the assay to detect CEA+EVs and/or CD73+EVs comprises treating the biological sample to remove soluble proteins and other interfering substances to produce a purified product. In embodiments, an assay to detect CEA+EVs in a biological sample comprises purifying the sample by, for instance, differential centrifugation, ultrafiltration, size-exclusion chromatography, immuno-affinity, precipitation, or a combination thereof, the biological sample before contacting the sample with a binding reagent or before detecting EVs. In embodiments, the binding reagent comprises an antibody or antigen binding fragment thereof, antigen, ligand, receptor, oligonucleotide, hapten, epitope, mimotope, or an aptamer.

In embodiments of the methods disclosed herein, an assay to detect CEA+EVs and/or CD73+EVs in a biological sample comprises contacting the biological sample, or a purification product therefrom, with a first binding reagent (i.e., a capture reagent) that binds to a first EV marker wherein the first binding reagent is immobilized to a surface (e.g., a bead or culture well), and contacting the biological sample or the purification product therefrom and/or the surface with a second binding reagent that binds to a second, different EV marker, wherein the second binding reagent comprises a detectable label. The first binding reagent may be immobilized to the surface before or after being contacted with the biological sample or the purification product therefrom. In embodiments, residual components in the biological sample or in the purification product therefrom, are removed from the surface after the first binding reagent is contacted with the biological sample or purification product, and after the first binding reagent is immobilized on the surface. The second binding reagent may be contacted with the biological sample or the purification product therefrom or the surface before or after the first binding reagent is contacted with the biological sample. In embodiments, the second binding reagent is contacted with the surface after the first binding reagent is contacted with the biological sample (or the purification product therefrom) and after the first binding reagent is immobilized on the surface. In embodiments, the second binding reagent is contacted with the biological sample or a purification product therefrom after the first binding reagent is contacted with the biological sample (or the purification product therefrom) but before the first binding reagent is immobilized. In embodiments, the second binding regent is contacted with the surface after the first binding reagent has been contacted with the biological sample (or the purification product therefrom), after the first binding reagent has been immobilized on the surface, and after residual, non-immobilized components of the biological sample (or the purification product therefrom) are removed from the surface (e.g., by washing the surface). In embodiments, the second binding regent is contacted with the surface after the first binding reagent is contacted with the biological sample (or the purification product therefrom) but before residual, non-immobilized components of the biological sample (or the purification product therefrom) are removed from the surface (e.g., by washing the surface).

Accordingly, in embodiments, performing an assay on a biological sample to detect CEA+EVs and/or CD73+EVs in the biological sample comprises contacting the biological sample (or a purification product therefrom) with a first binding reagent that binds to the CEA+EVs and/or CD73+EVs and immobilizing the first binding reagent on a surface to capture the CEA+EVs and/or CD73+EVs, removing the non-immobilized components of the biological sample or the purification product therefrom (e.g. by washing the surface), contacting the surface with a second binding reagent that binds to a different portion of the CEA+EVs and/or CD73+EVs and that comprises a detectable label, and detecting any detectable label that is bound to the captured EVs. In embodiments, the first binding reagent comprises an antibody or antibody fragment that binds to CEA and/or CD73 and the second binding reagent comprises an antibody or antibody fragment that binds to a tetraspanin (TTSP) such as, for example, CD81. Alternatively, the second binding reagent is a cocktail of binding reagents that bind to multiple different tetraspanins including CD81, CD9, and/or CD63. In embodiments, the first binding reagent comprises an antibody or antibody fragment that binds to a tetraspanin such as, for example, CD81 and the second binding reagent comprises an antibody or antibody fragment that binds to CEA and/or CD73. In embodiments, the first binding reagent comprises biotin and is immobilized on a surface that is coated with streptavidin.

In embodiments, biotinylated CEA or CD73 antibody (first binding reagent, aka capture reagent) is immobilized on a streptavidin-coated multi-well electrode coated plate such as an MSD GOLD™ 96-well Small Spot Streptavidin plate (Meso Scale Diagnostics, LLC.). A biological sample or a purified product therefrom is added to the plate wells, thereby contacting the sample with the immobilized first binding reagent. After incubating, the plates are washed and SULFO-TAG™ labeled CD81 antibody (second binding reagent) is added to the plate wells, thereby contacting the second binding reagent with the surface and the captured components (e.g., EVs) from the biological sample. After incubation, the plate is washed again and SULFO-TAG label is detected in the plate well, thereby detecting CEA+EVs and/or CD73+EVs from the biological sample. In embodiments, the CEA+EV capture assay may be part of a larger panel of assays for putative CRC-derived EVs. These multiple assays may be performed in the same well of a multiwell plate, (e.g. MSD U-PLEX 10-spot plate).

### CEA+/CD73+EVs

In embodiments of the methods disclosed herein, the methods comprise performing an assay to detect CEA+EVs that comprise both CEA and CD73 (CEA+/CD73+EVs). In embodiments, the method comprises a 3-marker proximity assay, such as a proximity ligation assay (PLA) or a proximity extension assay (PEA) that employs a first binding reagent that is a capture reagent, and two additional binding agents that are proximity probes (PP1 and PP2), wherein each of the three binding reagents binds to one of CEA, CD73, and a tetraspanin.

In embodiments, a three-marker assay for intact EVs is disclosed herein is used, using a first binding reagent (capture reagent) that binds CEA or CD73, and a second binding reagent (detect reagent) that binds CEA or CD73, and a third binding reagent (detect reagent) that binds a common EV marker (tetraspanin), such as CD81, CD63, CD81, or CD9.

Three-marker proximity assays and proximity probes are well known in the art. Proximity probes comprise an analyte binding domain (e.g., and antibody or antibody fragment) linked directly or indirectly to a functional domain (e.g., an oligonucleotide). Any of the 3-marker proximity assay formats disclosed in WO2019222708, WO2020086751, and WO2022051481 can be used to detect CEA+/CD73+EVs if the format uses binding reagents (i.e., a first binding reagent that is a capture reagent and two additional binding reagents that are proximity probes) that bind to CEA, CD73, and a tetraspanin. In embodiments, the binding reagents employed in an assay to detect CEA+/CD73+EVs employ any of the binding reagent configurations listed in Table 1.

TABLE 1

| | Capture reagent | PP1 | PP2 |
|---|---|---|---|
| | (first binding | (second binding | (third binding |
| | reagent) | reagent) | reagent) |
| Configuration 1 | CEA | CD81 or other | CD73 |
| | | TTSP | |
| Configuration 2 | CEA | CD73 | CD81 or other |
| | | | TTSP |
| Configuration 3 | CD73 | CEA | CD73 |
| Configuration 4 | CD73 | CD81 or other | CEA |
| | | TTSP | |
| Configuration 5 | CD81 or other | CD73 | CEA |
| | TTSP | | |
| Configuration 6 | CD81 or other | CEA | CD73 |
| | TTSP | | |
| Configuration 7 | CD73 | CD81 or other | CD73 |
| | | TTSP | |

In embodiments, the proximity probes PP1 and PP2 each comprise an analyte binding region and a functional oligonucleotide that contains a hybridization sequence that is complementary to the hybridization sequence of the partner proximity probe oligonucleotide. In embodiments, the PP1 and PP2 proximity probes engage in proximity extension when simultaneously bound to a common target.

In embodiments, the proximity probes PP1 and PP2 each comprise an analyte binding region and a functional oligonucleotide that contains a sequence that is complementary to an oligonucleotide template, such as a circular oligonucleotide template. In at least one of PP1 and PP2, the sequence that is complementary to the oligonucleotide template is a primer region that binds to the template and gets extended to produce a complement of the template (amplification). Any suitable amplification technique can be used to generate the extended sequence (or amplicon), including but not limited to, PCR (Polymerase Chain Reaction), LCR (Ligase Chain Reaction), and isothermal amplification methods, e.g., helicase-dependent amplification, rolling circle amplification (RCA), 3SR (Self-Sustained Synthetic Reaction), transcription mediated amplification (TMA), nucleic acid sequence-based amplification (NASBA), signal mediated amplification of RNA technology, strand displacement amplification (SDA), loop-mediated isothermal amplification of DNA (LAMP), isothermal multiple displacement amplification, single primer isothermal amplification, and circular helicase-dependent amplification. In embodiments, the assay/amplification technique is proximity ligation amplification (PLA) using RCA, which is known in the art, and disclosed in International Appl. No. PCT/US2015/030925, published as WO 2015/175856, which is incorporated herein by reference in its entirety.

In additional embodiments, the amplicon further comprises one or more detection sequences and the measuring step further comprises contacting the extended sequence with a plurality of labeled probes complementary to the one or more detection sequences. In embodiments, the labeled probe is labeled with a detectable label. Detectable labels are described further herein.

In embodiments, the amplicon remains localized on the surface following the amplification. In further embodiments of the methods of the invention, the amplicon remains bound to the surface after the amplification. This may be through the immobilized capture reagent that is bound to the EV that is bound to the proximity probes that generate the amplicon.

In an embodiment, RCA is used to make the amplicon because it has significant advantages in terms of sensitivity, multiplexing, dynamic range and scalability. Techniques for RCA are known in the art (see, e.g., Baner et al, Nucleic Acids Research, 26:5073 5078, 1998; Lizardi et al., Nature Genetics 19:226, 1998; Schweitzer et al. Proc. Natl. Acad. Sci. USA 97:10113 119, 2000; Faruqi et al., BMC Genomics 2:4, 2000; Nallur et al., Nucl. Acids Res. 29: ell8, 2001; Dean et al. Genome Res. 11:1095 1099, 2001; Schweitzer et al., Nature Biotech. 20:359 365, 2002; U.S. Pat. Nos. 6,054,274, 6,291,187, 6,323,009, 6,344,329 and 6,368,801). Several different variants of RCA are known, including linear RCA (LRCA) and exponential RCA (ERCA). RCA generates many thousands of copies of a circular template, with the chain of copies attached to the original target DNA, allowing for spatial resolution of target and rapid amplification of the signal. RCA facilitates (i) detection of single target molecules; (ii) amplification of signals from proteins as well as DNA and RNA; (iii) identifying the location of molecules that have been amplified on a solid surface; (iv) measurement of many different targets simultaneously; and (v) analysis of one or more targets in solution or solid phase. The spatial localization of immobilized RCA products is especially advantageous when conducting multiplexed binding assays in an array or particle-based format.

Thus, in embodiments, the method comprises using multiple (e.g., three) markers for the same CEA+EV to detect CEA+/CD73+EVs in a biological sample, the method comprising (a) contacting the biological sample or a purified product therefrom with (i) a first binding reagent immobilized on a surface; and (ii) first and second binding reagents, wherein the first and second binding reagents are proximity probes that each comprises an analyte binding region and an oligonucleotide; wherein each of the analyte binding regions is different from the other and each binds to one of CEA, CD73, and a tetraspanin such as CD81, wherein not more than one analyte binding region of the three different analyte binding regions binds to any of CEA, CD73, or the tetraspanin. In embodiments, the functional oligonucleotides of the first and second proximity probes bind to each other when bound to the same EV, after which proximity extension and amplification occur and an amplified product results. In embodiments, the functional oligonucleotides of the first and second proximity probes bind to a common template after which proximity ligation and amplification ensue. In embodiments, the amplified product is detected with a detectable probe.

In embodiments, the method comprises using multiple (e.g., three) markers for the same CEA+EV to detect CEA+/CD73+EVs in a biological sample, the method comprising contacting the biological sample or a purified product therefrom with a first binding reagent immobilized on a surface, and contacting the surface with first and second binding reagents, wherein the first and second binding reagents are proximity probes that each comprises an analyte binding region and an oligonucleotide; wherein each of the analyte binding regions is different from the other and each binds to one of CEA, CD73, and a tetraspanin such as CD81, wherein not more than one analyte binding region of the three different analyte binding regions binds to any of CEA, CD73, or the tetraspanin. In embodiments, the functional oligonucleotides of the first and second proximity probes bind to each other when bound to the same EV, after which proximity extension and amplification occur and an amplified product results. In embodiments, the functional oligonucleotides of the first and second proximity probes bind to a common template after which proximity ligation and amplification ensue. In embodiments, the amplified product is detected with a detectable probe.

In embodiments, the first binding reagent is a capture reagent that binds CEA, and the first and second proximity probes bind CD81 and CD73, respectively, or CD73 and CD81, respectively. The inventors surprisingly found that a 3-marker proximity assay using CEA capture reagent and CD81 and CD73 proximity probes reduces the signal from non-specifically bound EVs but doesn't diminish the signal from the colorectal cancer EVs specifically bound by CEA antibodies, leading to higher signal to background (see Example 2). Without being bound by theory, it is believed that the improved signal is due to a reduction in signal from irrelevant, non-specifically bound EVs when the more specific detection antibody conjugate mix of CD73 and CD81 binding reagents is used as compared to a detection mix containing CD63, CD81 and CD9 with CD81 binding reagents.

In embodiments, the method further comprises detecting signal emitted when both proximity probes are bound to the same immobilized EV. In embodiments, the first binding reagent (capture reagent) is immobilized on an electrode-coated surface, a SULFO-TAG ECL labeled probe is employed to detect amplification product from the proximity probes, and an electrochemiluminescent (ECL) signal is emitted and detected.

### III. Assay Detection and Detectable Labels

In embodiments, CEA+EVs and/or CD73+EVs are detected using electrochemiluminescence-based assay formats, e.g. electrochemiluminescence (ECL) based immunoassays. In some embodiments of the methods described herein, a CEA+EV and/or CD73+EVs is bound to a first binding reagent and a second binding reagent and the second binding reagent is labeled with a detectable label. In some embodiments of the methods described herein, a CEA+/CD73+EV is bound to a first binding reagent and two additional binding reagents that are proximity probes, and the proximity probes work together in a proximity process to produce an amplicon that is detected using a labeled probed that comprises a detectable label. In embodiments the detectable label can be induced to emit ECL. ECL-active species that have been used as ECL labels include, e.g., i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Species that participate with the ECL label in the ECL process are referred to herein as ECL coreactants. Commonly used coreactants include tertiary amines (e.g., see U.S. Pat. No. 5,846,485 and U.S. Provisional Application No. 62/787,892, filed on Jan. 3, 2019), oxalate, and persulfate for ECL from RuBpy and hydrogen peroxide for ECL from luminol (see, e.g., U.S. Pat. No. 5,240,863). In embodiments, the ECL coreactant is tripropylamine (TP A). In embodiments, the ECL coreactant is N-Butyldiethanolamine (BDEA). In embodiments, the ECL coreactant is N,N-dibutylethanolamine (DBAE). In embodiments, the ECL coreactant is included in a read buffer for the ECL assay. In embodiments, the read buffer comprises an ECL coreactant and a surfactant. In embodiments, the surfactant is TRITON X-100. In embodiments, the read buffer does not comprise TRITON X-100. In embodiments, the surfactant does not disrupt a surface of the surface marker displaying agent. In embodiments, the surfactant does not disrupt a lipid bilayer membrane. In embodiments, the surfactant does not disrupt a membrane of an EV. In embodiments, the surfactant is BRIJ, TWEEN, PLURONIC or KOLLIPHOR. In embodiments, the surfactant is TWEEN. In embodiments, the read buffer does not comprise a surfactant.

The light generated by ECL labels can be used as a reporter signal in diagnostic procedures (Bard et al., U.S. Pat. No. 5,238,808, herein incorporated by reference). For instance, an ECL label can be covalently coupled to a binding agent such as an antibody, nucleic acid probe, receptor or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants).

Alternatively, in embodiments, detectable labels used to detect CEA+EVs and/or CD73+EVs can be fluorescent species that can be used in single molecule fluorescence detection, e.g., fluorescence correlation spectroscopy, and/or fluorescence cross-correlation spectroscopy. Single molecule fluorescence detection comprises flowing an eluent that includes a detectable species through a capillary, focusing a light source on a volume within the capillary to create an interrogation zone and observing the interrogation zone with a light detector to detect the passage of fluorescent molecules through the interrogation zone. Alternatively, in embodiments labels use to detect analyte are colorimetric or luminescent.

### IV. Samples

In embodiments of the methods of the invention, EVs are detected in a sample that is a biological sample. In embodiments the biological sample is a fluid, secretion, or excretion. In embodiments, the biological sample is a purified mammalian fluid, secretion, or excretion. In embodiments, the mammalian fluid, secretion, or excretion is whole blood, plasma, serum, a liquid biopsy, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, or a wound secretion. Biological samples that may further include, but are not limited to, physiological samples and/or samples containing suspensions of cells, such as mucosal swabs, tissue aspirates, tissue homogenates, cell cultures, and cell culture supernatant, including cultures of eukaryotic and prokaryotic cells. In embodiments, cells or proteins are removed from a sample before detecting EVs. The cells and/or proteins may be removed by, for instance centrifugation or filtration.

In embodiments, the biological sample is obtained from a subject, e.g., a human. In embodiments, the biological sample comprises a plasma sample from a subject. In embodiments, the biological sample is obtained from a healthy subject. In embodiments, the biological sample is obtained from a subject suspected of having a colorectal tumor or suspected of having colorectal cancer (CRC). In embodiments, the subject is suspected of having late-stage CRC or stage IV CRC. In embodiments, the subject is diagnosed with having a colorectal tumor, CRC, late-stage CRC, or stage IV CRC. In embodiments, the subject is receiving treatment for CRC. In embodiments, the subject previously had CRC and was previously classified as being in remission.

“Stage IV” colorectal cancer as used herein includes Stages IV A, IV B, and IV C of the CRC TNM staging system established by the American Joint Committee on Cancer (AJCC) and encompasses colorectal cancer that (i) might or might not have grown through the wall of the colon or rectum (Any T), (ii) might or might not have spread to nearby lymph nodes (Any N), and (iii) has spread to 1 distant organ (such as the liver or lung) or distant set of lymph nodes, but not to distant parts of the peritoneum (the lining of the abdominal cavity) (M1a), or has spread to more than 1 distant organ (such as the liver or lung) or distant set of lymph nodes, but not to distant parts of the peritoneum (the lining of the abdominal cavity) (M1b), or has spread to distant parts of the peritoneum (the lining of the abdominal cavity), and may or may not have spread to distant organs or lymph nodes (M1c).

“Late stage” colorectal cancer as used herein includes Stages III and IV of the CRC TNM staging system established by the American Joint Committee on Cancer (AJCC).

### III. Surfaces

Methods of immobilizing a binding reagent to a surface are known to those of ordinary skill in the art. Suitable surfaces include the surfaces of macroscopic objects such as an interior surface of an assay container (e.g., test tubes, cuvettes, flow cells, FACS cell sorter, cartridges, wells in a multi-well plate, culture dishes, petri dishes, etc.), slides, assay chips (such as those used in gene or protein chip measurements), pins or probes, beads, filtration media, lateral flow media (for example, filtration membranes used in lateral flow test strips), etc. In embodiments, the surface is a bottom surface of a culture plate. In embodiments, the surface is a bottom surface of a well in a multi-well plate. In embodiments, the bottom surface comprises an electrode coating all or a portion of the bottom surface. In embodiments the electrode-coated surface is further coated with immobilized first binding reagent. In embodiments the electrode-coated surface is further coated with streptavidin. In embodiments the electrode-coated surface is further coated with streptavidin bound to biotin-conjugated antibodies or antibody fragments that are first binding reagents of an assay.

Suitable surfaces for use in the methods of the present invention are known in the art, including conventional surfaces from the art of binding assays. Suitable surfaces are disclosed, for example, in International Appl. No. PCT/US2015/030925, published as WO 2015/175856. Surfaces may be made from a variety of different materials including polymers (e.g., polystyrene and polypropylene), ceramics, glass, composite materials (e.g., carbon-polymer composites such as carbon-based inks).

Suitable surfaces also include particles (including but not limited to colloids or beads) commonly used in other types of particle-based assays e.g., magnetic, polypropylene, and latex particles, hydrogels, e.g. agarose, materials typically used in solid-phase synthesis e.g., polystyrene and polyacrylamide particles, and materials typically used in chromatographic applications e.g., silica, alumina, polyacrylamide, polystyrene. The materials may also be a fiber such as a carbon fibril. Microparticles may be inanimate or alternatively, may include animate biological entities such as cells, viruses, bacterium and the like. A particle used in the present method may be comprised of any material suitable for attachment to one or more capture or anchoring reagents, and that may be collected via, e.g., centrifugation, gravity, filtration or magnetic collection. A wide variety of different types of particles that may be attached to capture reagents are sold commercially for use in binding assays. These include nonmagnetic particles as well as particles comprising magnetizable materials which allow the particles to be collected with a magnetic field. In one embodiment, the particles are comprised of a conductive and/or semiconductive material, e.g., colloidal gold particles. The microparticles may have a wide variety of sizes and shapes. By way of example and not limitation, microparticles may be between 5 nanometers and 100 micrometers. Preferably microparticles have sizes between 20 nm and 10 micrometers. The particles may be spherical, oblong, rod-like, etc., or they may be irregular in shape.

The first binding reagents (i.e., capture reagents) employed in the methods disclosed herein can be immobilized by binding, directly or indirectly, to a surface or solid phase. The first binding reagents (i.e., capture reagents) employed in the methods disclosed herein can be immobilized by binding, directly or indirectly, to different discrete binding domains on one or more surfaces or solid phases, e.g., as in a binding array wherein the binding domains are individual array elements, or in a set of beads wherein the binding domains are the individual beads, such that discrete assay signals are generated on and measured from each binding domain. If capture reagents for different analytes are immobilized in different binding domains, the different analytes bound to those domains can be measured independently. In one example of such an embodiment, the binding domains are prepared by immobilizing, on one or more surfaces, discrete domains of capture reagents that bind analytes of interest. Optionally, the surface(s) may define, in part, one or more boundaries of a container (e.g., a flow cell, well, cuvette, etc.) which holds the sample or through which the sample is passed. In a preferred embodiment, individual binding domains are formed on electrodes for use in electrochemical or electrochemiluminescence assays. Multiplexed measurement of analytes on a surface comprising a plurality of binding domains using electrochemiluminescence has been used in the Meso Scale Diagnostics, LLC, MULTI-ARRAY® and SECTOR® Imager line of products (see, e.g., U.S. Pat. Nos. 10,201,812, 7,842,246 and 6,977,722, the disclosures of which are incorporated herein by reference in their entireties).

Still further, the first binding reagents (i.e., capture reagents) can be immobilized via direct or indirect binding to an electrode surface, which optionally includes different discrete binding domains, as described above. The electrode surface can be a component of a multi-well plate and/or a flow cell. Electrodes can comprise a conductive material, e.g., a metal such as gold, silver, platinum, nickel, steel, iridium, copper, aluminum, a conductive alloy, or the like. They may also include oxide coated metals, e.g., aluminum oxide coated aluminum. The electrode can include a working and counter electrodes which can be made of the same or different materials, e.g., a metal counter electrode and carbon working electrode. In one specific embodiment, electrodes comprise carbon based materials such as carbon, carbon black, graphitic carbon, carbon nanotubes, carbon fibrils, graphite, graphene, carbon fibers and mixtures thereof. In one embodiment, the electrodes comprise elemental carbon, e.g., graphitic, carbon black, carbon nanotubes, etc. Advantageously, they may include conducting carbon-polymer composites, conducting particles dispersed in a matrix (e.g. carbon inks, carbon pastes, metal inks, graphene inks), and/or conducting polymers. One specific embodiment of the invention is an assay module, preferably a multi-well plate, having electrodes (e.g., working and/or counter electrodes) that comprise carbon, e.g., carbon layers, and/or screen-printed layers of carbon inks.

In embodiments, the first binding reagent (capture reagent) comprises biotin and is immobilized to the surface which is coated with streptavidin wherein the immobilization is via biotin-streptavidin binding. In embodiments, the first binding reagent is immobilized to the surface via a direct thiol linkage. In embodiments, the first binding reagent (capture reagent) is immobilized to the surface via a pair of short complementary oligonucleotides (one attached to the surface, the other attached to the capture reagent) that form stable duplexes in common biological buffers but can be denatured in a low salt buffer, and modestly elevated temperature is used to allow the capture reagent, e.g., antibody to be released. In embodiments, a restriction site in the complementary oligonucleotides that is cleaved by a restriction endonuclease is used. In embodiments, the captured EVs are co-labeled with STAG-labeled detection antibodies as the second binding reagent.

In embodiments of the invention, the capture reagent (first binding reagent) binds to a first surface marker or protein on the CEA+EV and a second binding reagent binds to a second surface marker or protein on the CEA+EV.

In some embodiments, the disclosure provides a method as disclosed herein, further comprising communicating the method results to a healthcare provider. In some embodiments, the disclosure provides a method as disclosed herein, further comprising administering a colorectal cancer treatment regimen to the subject.

In embodiments, the present disclosure provides a method for administering a colorectal cancer treatment regimen to a subject in need thereof, comprising, evaluating the combined level of CEA+EVs and soluble CEA in a biological sample from the subject as compared to the combined level of CEA+EVs and soluble CEA in a reference, determining that the subject has CRC when the combined level of CEA+EVs and soluble CEA in a biological sample from the subject exceeds the combined level of CEA+EVs and soluble CEA in the reference; and administering the treatment regimen to the subject. In some embodiments, the disclosure provides a method as disclosed herein, further comprising, evaluating the combined level of soluble CEA and CEA+EVs in a biological sample from the subject as compared to the combined level of soluble CEA and CEA+EVs and in a reference comprises receiving one or more communications stating the measured levels of soluble CEA and CEA+EVs in a biological sample from the subject and the measured levels of soluble CEA and CEA+EVs in a reference.

In some embodiments, the disclosure provides a method as disclosed herein, further comprising, evaluating the combined level of soluble CEA and CEA+EVs in a biological sample from the subject comprises performing an assay on the biological sample from the subject to detect CEA+EVs in the biological sample and performing an assay on the biological sample from the subject to detect soluble CEA in the biological sample. In embodiments of the invention, the assay performed on the biological sample from the subject to detect CEA+EVs employs a first binding reagent that binds to CEA and a second binding reagent that binds to a tetraspanin. In embodiments of the invention, the biological sample is plasma, blood, serum, or a liquid biopsy. In embodiments of the invention, the CEA+EVs are CEA+/CD73+EVs. In some embodiments, performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes. In some embodiments, the reference is an equivalent biological sample from a normal subject that does not have colorectal cancer. In some embodiments, the disclosure provides a method as disclosed herein, comprising determining that the subject has stage III or stage IV CRC. In some embodiments, the treatment regimen is selected from the group consisting of administration of a targeted drug, chemotherapy, immunotherapy, radiation therapy, surgery, or a combination thereof.

In some embodiments, the disclosure provides methods as disclosed herein, wherein if the level of biomarkers at the second timepoint is 1.2 fold, 1.5 fold, 2 fold, 3 fold, 4 fold, 5 fold, or 10 fold greater than the first, then the subject is not responding favorably to treatment. In some embodiments, the disclosure provides methods as disclosed herein, wherein the level of biomarkers at the second timepoint is 1.2 fold, 1.5 fold, 2 fold, 3 fold, 4 fold, 5 fold, or 10 fold lower than the first, then the subject is responding favorably to treatment.

In embodiments the present disclosure provides a system comprising one or more data processors, and a non-transitory computer readable storage medium containing instruction which, when executed on the one or more data processors, cause the one or more data processors to perform part or all of the methods as disclosed herein. In embodiments, the present disclosure provides a non-transitory computer readable medium, comprising instructions configured to cause one or more data processors to perform part or all of the methods as disclosed herein.

In some embodiments, the present disclosure provides a non-transitory computer readable medium having stored thereon a computer program which, when executed by a computer system operably connected to an assay system configured to measure with a multiplexed binding assay, levels of a plurality of biomarkers comprising soluble CEA, soluble CD73, CEA+CD73+EV, CEA+EV, CD73+EV, or any combination thereof, in a sample from a human subject, causes the computer system to perform a method of predicting overall survival, progression free survival, duration of response, or a combination thereof in the subject receiving colorectal cancer treatment, the method comprising fitting the measured levels of the plurality of biomarkers to a response surface model as a function of overall survival, progression free survival, duration of response, or a combination thereof, computing a cost function, and selecting an overall survival, progression free survival, duration of response that minimizes the cost function, thereby identifying the overall survival, progression free survival, duration of response, or combination thereof.

In some embodiments, the present disclosure provides a method for managing treatment of a subject diagnosed with CRC, comprising receiving biomarker data corresponding to a biological sample obtained from the subject, computing a response score that predicts a likelihood of responsiveness to continued treatment using quantification data identified from the biomarker data wherein the biomarker comprises soluble CEA, soluble CD73, CEA+CD73+EV, CEA+EV, CD73+EV, or any combination thereof, and generating a treatment response output based on the response score. In some embodiments, generating the treatment response output comprises generating a predicted response to a treatment based on whether the response score is above a selected threshold. In further embodiments of the invention, the selected threshold is 1.0. The embodiments described herein provide a way of predicting treatment response with respect to survivability (e.g., overall survival, progression-free survival) at a baseline point in time prior to administration of the treatment.

In one or more embodiments, the response score is a probability that the subject will respond to the treatment based on a set of survival criteria. The set of survival criteria may include, for example, an overall survival greater than a selected period of time (e.g., 1 month, 3 months, 6 months, one year, two years, 100 days, 200 days, 500 days, etc.). The set of survival criteria may include, for example, a progression free survival greater than a selected period of time (e.g., 1 month, 3 months, 6 months, one year, two years, 100 days, 200 days, 500 days, etc.).

The present disclosure concerns embodiments for systems, methods, and compositions related to managing treatment for colorectal cancer.

### Examples

### Example 1—CEA+EVs and CD73+EVs in CRC Samples

After initially observing that some samples from late stage colorectal cancer (CRC) subjects exhibited high CEA-positive EV levels without high soluble CEA levels (4 of 15 samples exhibited high CEA+EV with low soluble CEA, data not shown), these observations were again observed in a subsequent cohort of samples from 23 late stage CRC subjects and 16 matched controls (5 of 23 samples exhibited high CEA+EV with low soluble CEA, data not shown). An additional, larger cohort of patient plasma samples from subjects with various stages of CRC and normal controls (~25 normal controls, ~25 stage I, ~25 stage II, ~25 Stage III, ~25 stage IV) was then tested using ultrasensitive ECL assays for CEA positive EVs and CD73+EVs, and an ECL assay for soluble CEA (MSD R-PLEX® CEA assay, Meso Scale Diagnostics, LLC.).

### Sample Preparation:

Samples were coded and randomized so as to be blinded to the operator. An aliquot of each sample was taken for measuring soluble proteins. The remaining sample was subjected to an EV purification protocol using CaptoCore400 to remove the majority of soluble proteins, which could act as interferants in the subsequent assays. CaptoCore400 resin slurry was washed twice with DPBS. Plasma was diluted 1:3 in DPBS, then mixed with washed resin and incubated with shaking for 1 hour. Unbound EVs were eluted by centrifugation in a fritted plate, while most of the soluble proteins were retained by the resin. The eluted fraction was used for EV assays.

### Assay Plate Preparation:

MSD U-PLEX® multi-spot ECL assay plates (Meso Scale Diagnostics, LLC.) were prepared by immobilizing first binding reagents (capture reagents) comprising antibodies to CEA, CD73, or IgG1 negative control antibodies to spots on the inner bottom surface of the plate wells using U-PLEX® reagents (Meso Scale Diagnostics, LLC.). After immobilization, each well contained spots with immobilized CEA capture reagent, spots with immobilized CD73 capture reagent, and at least one spot with the negative control immobilized capture reagent.

### Assays:

CEA+EVs—25 μL diluent 52 was added to each well, followed by 25 μL of a heat purified EV sample from plasma. Plates were incubated with shaking for 1 hour to capture EVs. Following the EV capture step, plates were washed to remove unbound EVs, then a mixture of second and third binding reagents (detection antibody conjugates) was added. The detection antibody conjugates comprised a mixture of proximity probes comprising CD63, CD81 and CD9 analyte binding domains (PP1 conjugates) and CD81 analyte binding domains (PP2 conjugates), ensuring that only EVs with at least one copy of CD81 were detected. These detection conjugates for ultrasensitive assays were added to the plate wells to label the captured EVs and incubated for 1 hour with shaking. Unbound detection antibody conjugates were then washed away and rolling circle amplification (RCA) was performed to amplify the signal from labeled EVs prior to reading on an MSD SECTOR® Imager (Meso Scale Diagnostics, LLC.).

Soluble CEA—Soluble CEA was measured according to the manufacturer's instructions in the MSD R-PLEX® CEA assay product insert.

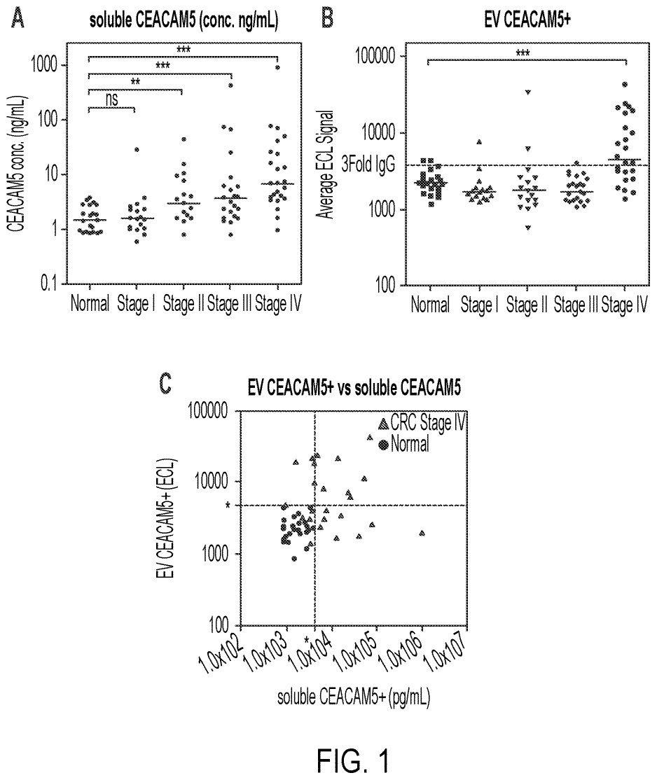

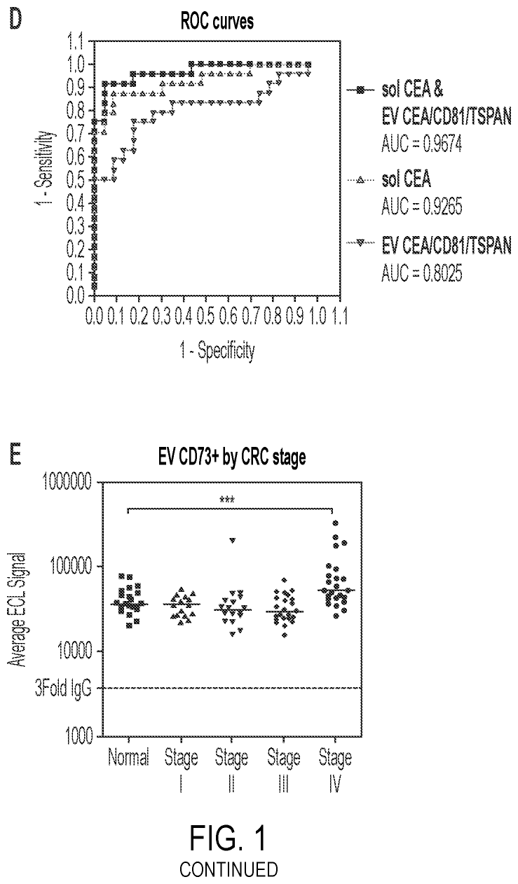

### Results (FIG. 1):

CEA-positive EVs were elevated in most late-stage patients and some earlier stage patients relative to the non-cancer controls (FIG. 1B). CD73-positive EVs were also elevated in most late-stage patients, though the discrimination between cancer and non-cancer patients was not as good as CEA-positive EVs (FIG. 1E). The level of CEA-positive EVs and CD73-positive EVs were highly correlated. Soluble CEA, on the other hand, was not as well correlated with CEA-positive EVs (FIG. 1C). As seen in the two cohorts measure previously, a subset of CRC patients with elevated CEA-positive EVs, relative to non-cancer subjects did not exhibit elevated soluble CEA. Several earlier stage patients with high CEA+EVs but not high soluble CEA were also observed. These results indicate that measuring CEA+EVs likely identifies a different subset of CRC patients than the subset identified by measuring soluble CEA, and that the combination of these two measurements is likely to identify CRC patients with higher sensitivity than either measurement alone.

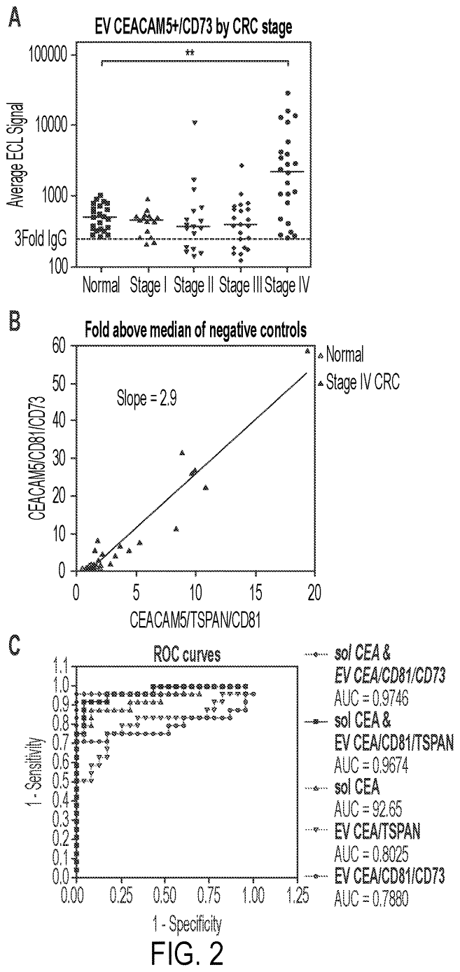

### Example 2—Double Positive CEA+/CD73+EVs in CRC Samples (FIG. 2)

Since CD73+EVs and CEA+EVs were highly correlated (see Example 1), it was postulated that these markers may be colocalized on the same EVs. A 3-antibody ultrasensitive ECL proximity assay similar to Example 1 was developed to detect the CEA+/CD73+double positive population of EVs (“CEA+/CD73+EVs”) in a sample. This assay comprised a first binding reagent that was a CEA capture antibody, and two additional binding reagents that were proximity probes: CD81 PP1 and CD73 PP2. Using this assay, it was confirmed that CEA+/CD73+EVs were present in conditioned medium from the colorectal cancer cell line Difi (data not shown) and in a small cohort of samples from CRC subjects (data not shown). Similar results were produced when reversing the position of the two proximity probe antibodies (CD73 PP1 and CD81 PP2).

This assay was then applied to the same cohort of CRC samples as in Example 1 and CEA+/CD73+EVs were observed in the same set of late-stage patients as the CEA+EVs in Example 1 with similarly high ECL signals in the cancer subjects (FIG. 2A). The signal in non-cancer subjects was reduced markedly when measuring the CEA+/CD73+EVs relative to the CEA+EV measurements from Example 1. This resulted in an improvement of signal to background of approximately 3-fold. Similar results were observed in the CRC patients and controls when reversing the position of the two antibodies (CD73 PP1 and CD81 PP2).

### Example 3—Survival Analysis of CaboMab and X-TRAP Cohorts Using EV-Associated and Soluble Forms of CEA and CD73

### Sample Description

Samples were analyzed from two cohorts of late-stage refractory colorectal cancer. The first cohort was from the CaboMab trial. Patients were treated with Panitumumab (Anti-EGFR) and Cabozantinib (kinase inhibitor targeting VEGFR and c-MET. We assayed samples taken immediately before treatment and ~3 weeks after the first treatment. The second cohort was from the X-TRAP trial. Patients were treated with capecitabine (ChemoT) and ziv-aflibercept, a recombinant fusion protein containing extracellular domain of VEGFR1 and VEGFR2, which binds to VEGF-A, VEGF-B and PIGF. We assayed samples taken immediately before treatment and ~3 weeks after the first treatment.

### Raw Measurements

At each timepoint we measured soluble CD73 (sCD73), and soluble CEA (sCEA, also called CEACAM5) using standard ECL sandwich assays, EVs presenting CEA on their outer surface (CEA+EVs), EVs presenting CD73 on their outer surface (CD73+EVs), using ultrasensitive ECL assays for intact EVs, EVs presenting both CEA and CD73 on their outer surface (CEA+CD73+EVs,) using an ultrasensitive ECL assay for intact EVs having a CEA capture antibody and CD73 detection antibody, and EVs presenting more than one copy of CD73 on their surface (CD73+CD73+EVs) using an ultrasensitive assay for intact EVs using a CD73 capture antibody and a CD73 detection antibody. Raw ECL counts measured at the first and second timepoints were log 2 transformed before making statistical comparisons. We also calculated the change in each measured biomarker between timepoint one and two. These values were not transformed before making statistical comparisons.

### Cox Proportional Hazard Model

We used a Cox model to investigate the association between overall survival time (OS), progression free survival time (PFS) and duration of treatment response (DR) and the measured concentrations of each biomarker at the first timepoint, the second timepoint, and the initial trajectory (Biomarker value at Second timepoint divided by value at first timepoint). First, we performed univariate Cox analysis for each biomarker variable and outcome variable: OS, PFS in the CaboMab cohort and OS, PFS and DR in X-TRAP.

Table 2 below shows biomarkers that were significant predictors of each of the measured outcomes in each of the two cohorts. The hazard ratio is the increase in hazard for a unit change in the biomarker. Since these are log 2 transformed, this is the increase in hazard for a 2-fold change in the biomarker. Significance is calculated using the Wald test. The Wald statistic value and associated p-value are listed in Table 3.

TABLE 2
Univariate Cox Regressions

| | Out- | Time- | | Hazard | | Wald | |
|---|---|---|---|---|---|---|---|
| Cohort | come | point | Biomarker | Ratio | (95% CI) | Stat | p-value |
| CaboMab | OS | 1 | sCD73 | 2 | 1.4-3 | 12 | 0.00061 |
| CaboMab | OS | 1 | SCEA | 1.3 | 1.1-1.6 | 11 | 0.00075 |
| CaboMab | OS | 1 | CEA_Evs | 1.5 | 1.2-1.8 | 16 | 5.60E−05 |
| CaboMab | OS | 1 | CD73_Evs | 2.1 | 1.3-3.4 | 9 | 0.0027 |
| CaboMab | OS | 1 | CD73CD73_Evs | 1.5 | 1.2-2 | 10 | 0.0014 |
| CaboMab | OS | 1 | CEACD73_Evs | 1.3 | 1.1-1.6 | 12 | 0.00045 |
| CaboMab | OS | 2 | SCD73 | 2.2 | 1.4-3.6 | 11 | 0.0012 |
| CaboMab | OS | 2 | sCEA | 1.3 | 1.1-1.6 | 12 | 0.00062 |
| CaboMab | OS | 2 | CEA_Evs | 1.8 | 1.4-2.5 | 18 | 2.20E−05 |
| CaboMab | OS | 2 | CD73_Evs | 2.5 | 1.4-4.8 | 8.4 | 0.0037 |
| CaboMab | OS | 2 | CD73CD73_Evs | 1.9 | 1.4-2.6 | 14 | 0.00018 |
| CaboMab | OS | 2 | CEACD 73_Evs | 1.5 | 1.2-1.9 | 16 | 5.60E−05 |
| X-TRAP | OS | 1 | sCD73 | 1.4 | 1.1-1.8 | 5.9 | 0.015 |
| X-TRAP | OS | 1 | CEA_Evs | 1.3 | 1.1-1.5 | 6.6 | 0.01 |
| X-TRAP | OS | 1 | CD73CD73_Evs | 1.3 | 1.1-1.6 | 6.4 | 0.011 |
| X-TRAP | OS | 1 | CEACD73_Evs | 1.2 | 1.1-1.4 | 7.7 | 0.0055 |
| X-TRAP | OS | 2 | SCEA | 1.1 | 1-1.3 | 3.9 | 0.049 |
| X-TRAP | OS | 2 | CEA_Evs | 1.3 | 1-1.6 | 5.9 | 0.015 |
| X-TRAP | OS | 2 | CD73CD73_Evs | 1.3 | 1.1-1.6 | 6.8 | 0.0089 |
| X-TRAP | OS | 2 | CEACD73_Evs | 1.2 | 1.1-1.5 | 7.4 | 0.0064 |
| CaboMab | PFS | 1 | sCD73 | 1.7 | 1.2-2.5 | 8.3 | 0.004 |
| CaboMab | PFS | 1 | CEA_Evs | 1.4 | 1.1-1.7 | 9.3 | 0.0023 |
| CaboMab | PFS | 1 | CD73CD73_Evs | 1.4 | 1.1-1.8 | 7.6 | 0.0057 |
| CaboMab | PFS | 1 | CEACD73_Evs | 1.3 | 1.1-1.5 | 9.4 | 0.0022 |
| CaboMab | PFS | 2 | sCD73 | 1.6 | 1.1-2.5 | 5.2 | 0.023 |
| CaboMab | PFS | 2 | CEA_Evs | 1.4 | 1.1-1.8 | 6.6 | 0.01 |
| CaboMab | PFS | 2 | CD73CD73_Evs | 1.5 | 1.2-2 | 9.8 | 0.0018 |
| CaboMab | PFS | 2 | CEACD73_Evs | 1.4 | 1.1-1.6 | 11 | 0.0011 |
| CaboMab | OS | Init Traj | CEACD73_Evs | 2.3 | 1.1-4.9 | 4.6 | 0.032 |
| X-TRAP | OS | Init Traj | CEACD73_Evs | 1.8 | 1.1-2.9 | 4.6 | 0.032 |
| X-TRAP | DR | Init Traj | CD73_Evs | 2.2 | 1.1-4.5 | 5 | 0.026 |
| X-TRAP | DR | Init Traj | CEACD73_Evs | 1.8 | 1.1-3.1 | 5.1 | 0.024 |

For each biomarker that was a significant predictor, we explored combinations using multivariate Cox regression. Each multivariate model that predicted the outcome with a higher significance than the best univariate component for that outcome is listed in the table below.

TABLE 3
Multivariate Cox Regression

| | Out | Time | Bio- | exp(Co- | | Likelihood | |
|---|---|---|---|---|---|---|---|
| Cohort | come | point | marker | efficient) | (95% CI) | Ratio test | p-value |
| CaboMab | OS | 1 | sCEA | 1.244 | 1.035-1.496 | 20.52 | 3.00E−05 |
| | | | CEA Evs | 1.38 | 1.113-1.711 | | |
| CaboMab | OS | 2 | sCEA | 1.691 | 1.249-2.289 | 23.61 | 7.00E−06 |
| | | | CEA Evs | 1.238 | 1.027-1.493 | | |

### Kaplan Meier Analysis

In order to apply Kaplan Meier (KM) analysis we transformed the continuous variables of biomarker concentrations or signals into categorical variables. Samples above the median were assigned the value “High,” whereas those at or below the median were assigned the value “Low.” The trajectories were also categorized “low” for those as at or below the median and “high” for those above the median. We plotted the KM curves below for each individual biomarker shown to be significantly predictive by Cox Regression at time point 1, timepoint 2 or initial trajectory. Since the multivariate model of sCEA and CEA_EVs was highly predictive, we combined these two variable into one using a logical OR, that is, if either value was “high,” the combined categorical value was assigned “high”.

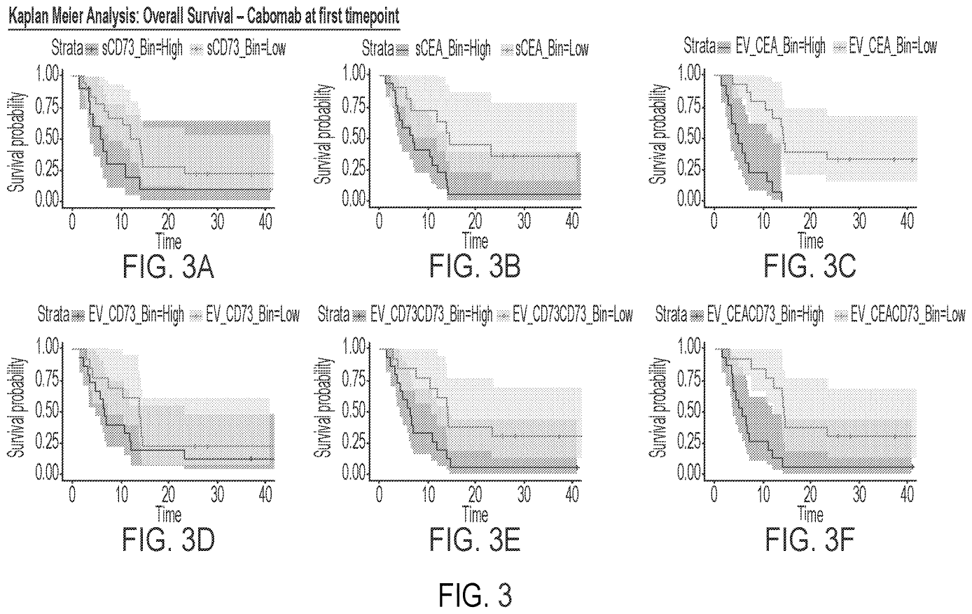

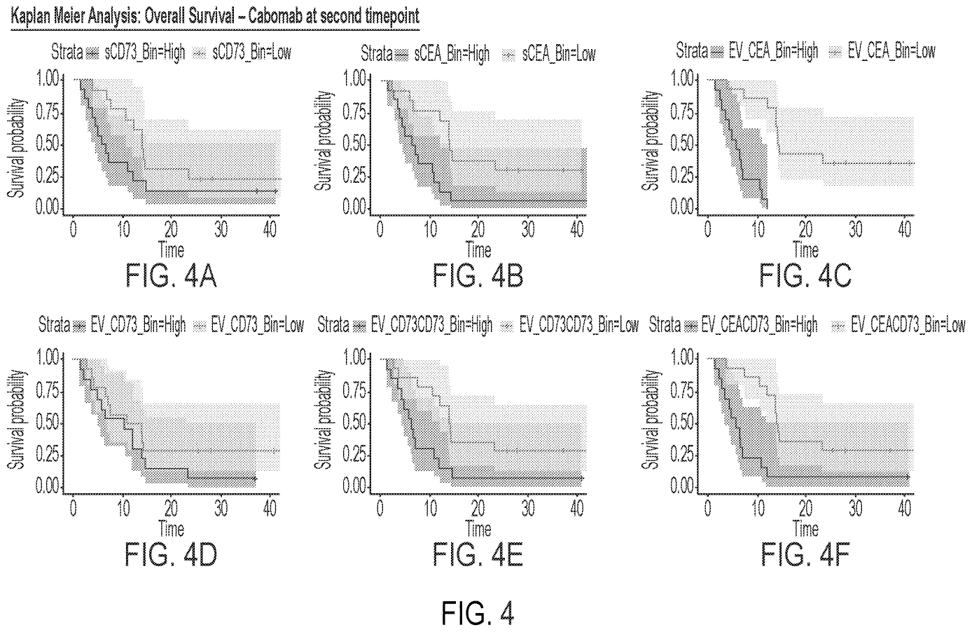

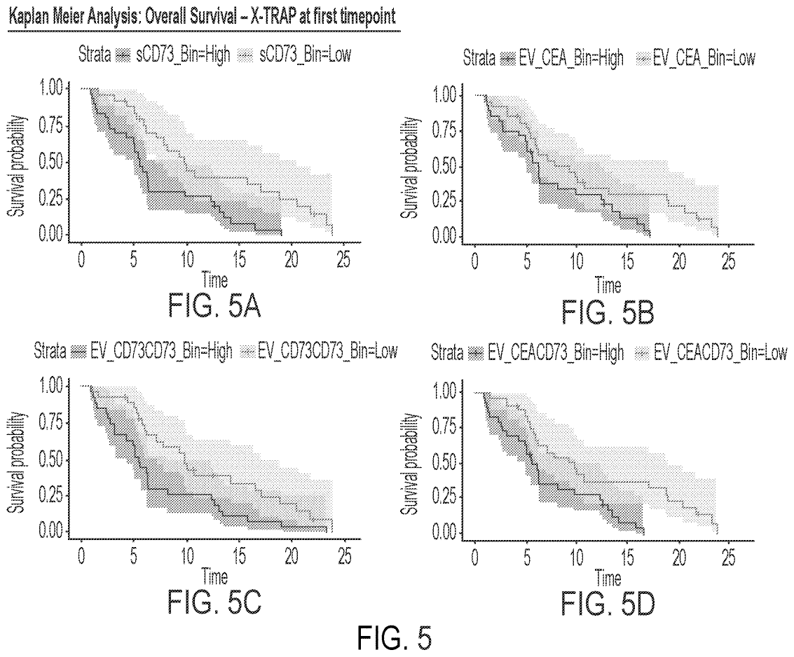

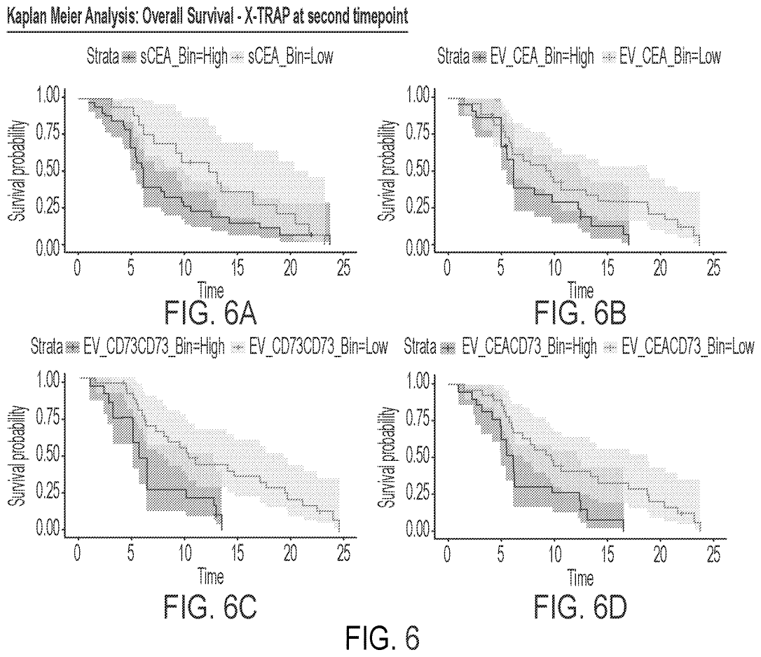

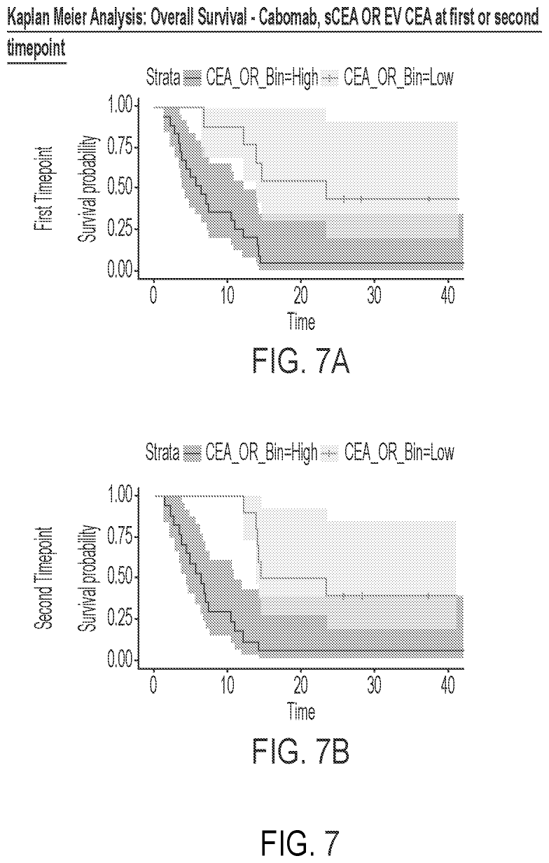

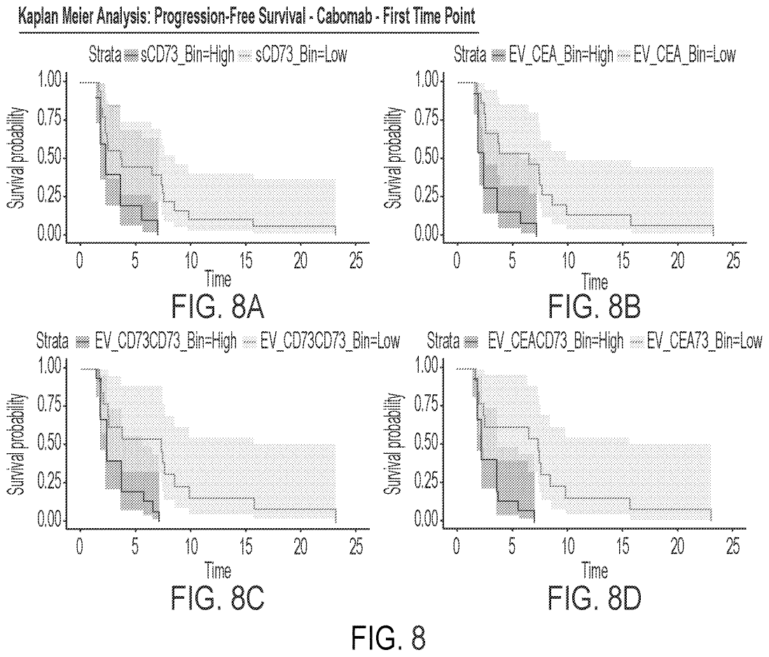

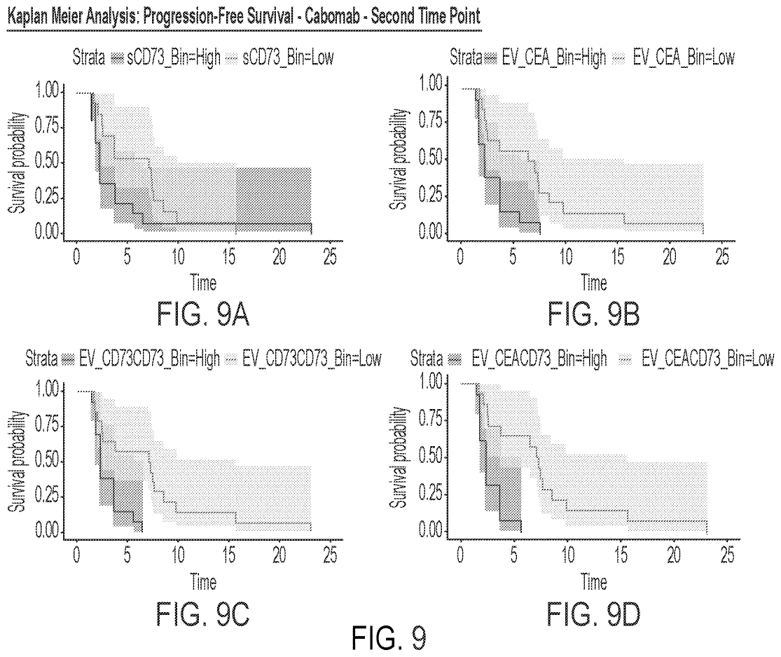

The level of EVs that presented CEA, denoted as EV_CEA in FIGS. 3-9 were most predictive of Overall survival in the X-TRAP cohort, where there is a clear separation in survival between the patients with low EV_CEA and those with high EV_CEA, particularly around 5-15 months. These were much more predictive of OS or PFS than soluble CEA.

What is claimed is:

1. A method for detecting colorectal tumor derived EVs in a sample from a subject suspected of having a colorectal tumor or suspected of having colorectal cancer (CRC), the method comprising detecting CEA+/CD73+double positive EVs in the sample.

2. The method of claim 1, wherein the sample is a plasma, whole blood, serum, or a liquid biopsy.

3. The method of claim 1, wherein the subject is suspected of having late-stage CRC.

4. The method of claim 3, wherein the subject is suspected of having stage IV CRC.

5. The method of any of claims 1-4, wherein detecting CEA+/CD73+double positive EVs comprises performing a 3-marker proximity assay on the sample or on a purification thereof.

6. A method for diagnosing colorectal cancer (CRC) or the presence of a colorectal tumor in a subject suspected of having CRC or suspected of having a colorectal tumor, the method comprising:
performing an assay on a biological sample from the subject to detect CEA+EVs in the biological sample;
performing an assay on the biological sample from the subject to detect soluble CEA in the biological sample;
determining whether the combined level of soluble CEA and CEA+EVs in the biological sample from the subject exceeds the combined level of CEA+EVs and soluble CEA in a reference; and
diagnosing the subject as having CRC if the combined level of soluble CEA and CEA+EVs in the biological sample from the subject exceeds the combined level of CEA+EVs and soluble CEA in the reference.

7. The method of claim 6, wherein the biological sample is plasma, blood, serum, or a liquid biopsy.

8. The method of claim 6, wherein the CRC is stage IV CRC.

9. The method of claim 6, wherein the assay to detect CEA+EVs employs a first binding reagent that binds to CEA and a second binding reagent that binds to a tetraspanin.

10. The method of claim 6, wherein the CEA+EVs are CEA+/CD73+EVs.

11. The method of claim 10, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

12. The method of claim 6, wherein the reference is an equivalent biological sample from a healthy subject that does not have colorectal cancer.

13. A method for staging CRC in a subject, said method comprising:
performing an assay on a biological sample from the subject to detect CEA+EVs in the biological sample;
performing an assay on the biological sample from the subject to detect soluble CEA in the biological sample;
determining whether the combined level of soluble CEA and CEA+EVs in the biological sample from the subject exceeds the combined level of CEA+EVs and soluble CEA in a reference; and
classifying the subject as having stage IV CRC if the combined level of soluble CEA and CEA+EVs in the biological sample from the subject exceeds the combined level of CEA+EVs and soluble CEA in the reference.

14. The method of claim 13, wherein the biological sample is plasma, blood, serum, or a liquid biopsy.

15. The method of claim 13, wherein the assay to detect CEA+EVs employs a first binding reagent that binds to CEA and a second binding reagent that binds to a tetraspanin.

16. The method of claim 13, wherein the CEA+EVs are CEA+/CD73+EVs.

17. The method of claim 16, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

18. The method of claim 13, wherein the reference is an equivalent biological sample from a healthy subject that does not have colorectal cancer.

19. A method for determining a prognosis of a subject diagnosed with CRC or suspected of having CRC, the method comprising:
performing an assay on a biological sample from the subject to detect CEA+EVs in the biological sample, performing an assay on the biological sample from the subject to detect soluble CEA in the biological sample;
determining whether the combined level of soluble CEA and CEA+EVs in the biological sample from the subject exceeds the combined level of CEA+EVs and soluble CEA in a reference; and
determining that the subject has a poor prognosis if the combined level of soluble CEA and CEA+EVs in the biological sample from the subject exceeds the combined level of CEA+EVs and soluble CEA in the reference.

20. The method of claim 19, wherein the biological sample is plasma, blood, serum, or a liquid biopsy.

21. The method of claim 19, wherein the assay to detect CEA+EVs employs a first binding reagent that binds to CEA and a second binding reagent that binds to a tetraspanin.

22. The method of claim 19, wherein the CEA+EVs are CEA+/CD73+EVs.

23. The method of claim 22, wherein performing an assay on a biological sample from the subject to detect CEA+EVs comprises:
performing a 3-marker proximity assay that employs a first binding reagent that binds CEA, and a second and third binding reagent that bind CD81 and CD73, respectively;
wherein the first binding reagent is a capture reagent and the second and third binding reagents are proximity probes.

24. The method of claim 19 wherein the reference is an equivalent biological sample from a healthy subject that does not have colorectal cancer.

25. A method for aiding in the detection of CRC or a colorectal tumor in a subject suspected of having CRC or a colorectal tumor, the method comprising:
performing an assay on a biological sample from the subject to detect CEA+EVs in the biological sample; and
performing an assay on the biological sample from the subject to detect soluble CEA in the biological sample and comparing the combined level of soluble CEA and CEA+EVs in said biological sample with the combined level of soluble CEA and CEA+EVs in a reference;
wherein a combined level of CEA+EVs and soluble CEA in the biological sample that is greater than the combined level of CEA+EVs and soluble CEA in the reference aids in detecting CRC or a colorectal tumor in the subject.

26. The method of claim 25, wherein the biological sample is plasma, blood, serum, or a liquid biopsy.

27. The method of claim 25, wherein the CRC is stage IV CRC.

28. The method of claim 25, wherein the assay to detect CEA+EVs employs a first binding reagent that binds to CEA and a second binding reagent that binds to a tetraspanin.

29. The method of claim 25, wherein the CEA+EVs are CEA+/CD73+EVs.

30. The method of claim 29, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

31. The method of claim 25, wherein the reference is an equivalent biological sample from a healthy subject that does not have colorectal cancer.

32. A method for aiding in the staging of CRC in a subject, said method comprising:
performing an assay on a biological sample from the subject to detect CEA+EVs in the biological sample; and
performing an assay on the biological sample from the subject to detect soluble CEA in the biological sample, and comparing the combined level of soluble CEA and CEA+EVs in said biological sample with the combined level of soluble CEA and CEA+EVs in a reference;
wherein a combined level of CEA+EVs and soluble CEA in the biological sample that is greater than the combined level of CEA+EVs and soluble CEA in the reference aids in classifying the subject as having stage IV CRC.

33. The method of claim 32, wherein the biological sample is plasma, blood, serum, or a liquid biopsy.

34. The method of claim 32, wherein the assay to detect CEA+EVs employs a first binding reagent that binds to CEA and a second binding reagent that binds to a tetraspanin.

35. The method of claim 32, wherein the CEA+EVs are CEA+/CD73+EVs.

36. The method of claim 35, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

37. The method of claim 32, wherein the reference is an equivalent biological sample from a healthy subject that does not have colorectal cancer.

38. A method for assisting in determining a prognosis of a subject diagnosed with CRC or suspected of having CRC, the method comprising:
performing an assay on a biological sample from the subject to detect CEA+EVs in the biological sample; and
performing an assay on the biological sample from the subject to detect soluble CEA in the biological sample, and comparing the combined level of soluble CEA and CEA+EVs in said biological sample with the combined level of soluble CEA and CEA+EVs in a reference;
wherein a combined level of CEA+EVs and soluble CEA in the biological sample that is greater than the combined level of CEA+EVs and soluble CEA in the reference aids in determining that the subject has a poor prognosis.

39. The method of claim 38, wherein the biological sample is plasma, blood, serum, or a liquid biopsy.

40. The method of claim 38, wherein the assay to detect CEA+EVs employs a first binding reagent that binds to CEA and a second binding reagent that binds to a tetraspanin.

41. The method of claim 38, wherein the CEA+EVs are CEA+/CD73+EVs.

42. The method of claim 41, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

43. The method of claim 38 wherein the reference is an equivalent biological sample from a healthy subject that does not have colorectal cancer.

44. A method for monitoring CRC recurrence in a subject previously classified as being in CRC remission, said method comprising:
performing an assay on a biological sample from the subject to detect CEA+EVs in the biological sample; and
performing an assay on the biological sample from the subject to detect soluble CEA in the biological sample, and comparing the combined level of soluble CEA and CEA+EVs in said biological sample with the combined level of soluble CEA and CEA+EVs in a reference;
wherein a combined level of CEA+EVs and soluble CEA in the biological sample that is greater than the combined level of CEA+EVs and soluble CEA in the reference aids in determining that CRC has recurred in the subject.

45. The method of claim 44, wherein the biological sample is plasma, blood, serum, or a liquid biopsy.

46. The method of claim 44, wherein the assay to detect CEA+EVs employs a first binding reagent that binds to CEA and a second binding reagent that binds to a tetraspanin.

47. The method of claim 44, wherein the CEA+EVs are CEA+/CD73+EVs.

48. The method of claim 47, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

49. The method of claim 44, wherein the reference is an equivalent biological sample from a healthy subject that does not have colorectal cancer.

50. A method for detecting, diagnosing, staging, monitoring changes or recurrence, or predicting response of a colorectal tumor to a treatment in a subject comprising:
(a) performing an assay on a biological sample from the subject to detect a level of at least one specific population of EVs in the biological sample;
(b) comparing the level of specific EVs to a reference level; and
(c) determining the presence, diagnosis, stage, progression, or response of the tumor based on the level of EVs in one or more samples relative to the reference.

51. The method of claim 50, wherein the specific population of EVs is defined by the presence of at least one surface marker selected from CEA (CEACAM5), CD73 or a combination thereof.

52. The method of claim 51 or 52, further comprising performing one or more assays on at least one biological sample from the subject to detect a soluble biomarker.

53. The method of claim 53, wherein the soluble biomarker is CEA, CD73, or a combination thereof.

54. The method of claim 53, wherein the surface marker defining the EV population and the soluble biomarker are substantially the same protein.

55. The method of claim 50, wherein the reference level is determined from a biological sample from a second subject without a colorectal tumor, from a biological sample from the same subject, or from a synthetic sample.

56. The method of claim 55, wherein the synthetic sample is a calibrator.

57. The method of any of claims 50-56, wherein the monitoring changes comprises determining the colorectal tumor size or stage between two timepoints.

58. The method of any of claims 50-56, wherein the monitoring changes comprises determining overall survival, progression free survival, response to treatment, or duration of response to treatment.

59. The method of any of claims 50-58, wherein the specific population of EVs is identified by the presence of CEA (CEACAM5), and wherein the method comprises predicting overall survival, progression-free survival, duration of response to treatment, or a combination thereof.

60. The method of claim 50-58, wherein the specific population of EVs is identified by the presence of CD73, and wherein the method comprises predicting overall survival, progression-free survival, duration of response to treatment, or a combination thereof.

61. The method of claim 50, wherein step (a) comprises performing a 3-marker proximity assay comprising a first detection reagent that binds CEA+EVs or CD73+EVs, and a second and third binding reagent that binds CEA+EVs, CD73EVs, or a tetraspanin, wherein the first binding reagent is a capture reagent and the second and third binding reagents are detection reagents.

62. The method of claim 61, wherein the tetraspanin is CD63, CD81, or CD9.

63. A method for monitoring response to CRC treatment in a subject receiving CRC treatment, the method comprising:
performing an assay on a first biological sample obtained from the subject before CRC treatment administration to detect CEA+EVs in the first biological sample;
performing an assay on said first biological sample obtained from the subject before CRC treatment administration to detect soluble CEA in the first biological sample;
performing an assay on a second biological sample obtained from the subject after CRC treatment administration to detect CEA+EVs in said second biological sample; and
performing an assay on said second biological sample obtained from the subject after CRC treatment administration to detect soluble CEA in said second biological sample;
wherein a combined level of CEA+EVs and soluble CEA in the second biological sample that is greater than or equal to the combined level of CEA+EVs and soluble CEA in the first biological sample indicates that the subject is not responding favorably to said treatment; and
wherein the second biological sample is obtained from the subject at least 7 days to 6 months after administration of said CRC treatment to said subject.

64. A method for monitoring response to CRC treatment in a subject receiving CRC treatment, the method comprising:
performing an assay on a first biological sample obtained from the subject before CRC treatment administration to detect CEA+CD73+EVs in the first biological sample; and
performing an assay on a second biological sample obtained from the subject after CRC treatment administration to detect CEA+CD73+EVs in the second biological sample;
wherein a level of CEA+CD73+EVs in the second biological sample that is greater than or equal to a level of CEA+CD73+EVs in the first biological sample indicates that the subject is not responding favorably to said treatment; and
wherein the second biological sample is obtained from the subject 7 days to 6 months after administration of the CRC treatment to said subject.

65. The method of claim 64, further comprising:
performing an assay on the first biological sample to detect CD73+EVs;
performing an assay on the second biological sample to detect CD73+EVs;
wherein a combined level of CEA+CD73+EVs and CD73+EVs in the second biological sample that is greater than or equal to a level of CEA+CD73+EVs and CD73+EVs in the first biological sample indicates that the subject is not responding favorably to said treatment; and
wherein the second biological sample is obtained from the subject 7 days to 6 months after administration of the CRC treatment to the subject.

66. The method of any of claim 63 or 64, wherein biological sample is plasma, blood, serum, or a liquid biopsy.

67. The method of any of claim 63 or 64, wherein the subject was previously diagnosed with stage IV CRC.

68. The method of claim 63, wherein the assay to detect CEA+EVs employs a first binding reagent that binds to CEA and a second binding reagent that binds to a tetraspanin.

69. The method of claim 63, wherein the CEA+EVs are CEA+/CD73+EVs.

70. The method of any of claim 63 or 64, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

71. The method of any of claims 1-70 further comprising communicating the method results to a healthcare provider.

72. The method of any of claims 1-70 further comprising administering a colorectal cancer treatment regimen to the subject.

73. A method for administering a colorectal cancer treatment regimen to a subject in need thereof, the method comprising:
evaluating the combined level of CEA+EVs and soluble CEA in a biological sample from the subject as compared to the combined level of CEA+EVs and soluble CEA in a reference;
determining that the subject has CRC when the combined level of CEA+EVs and soluble CEA in a biological sample from the subject exceeds the combined level of CEA+EVs and soluble CEA in the reference; and
administering the treatment regimen to the subject.

74. The method of claim 73, wherein evaluating the combined level of soluble CEA and CEA+EVs in a biological sample from the subject as compared to the combined level of soluble CEA and CEA+EVs and in a reference comprises receiving one or more communications stating the measured levels of soluble CEA and CEA+EVs in a biological sample from the subject and the measured levels of soluble CEA and CEA+EVs in a reference.

75. The method of claim 73, wherein evaluating the combined level of soluble CEA and CEA+EVs in a biological sample from the subject comprises performing an assay on the biological sample from the subject to detect CEA+EVs in the biological sample and performing an assay on the biological sample from the subject to detect soluble CEA in the biological sample.

76. The method of claim 75 wherein the assay performed on the biological sample from the subject to detect CEA+EVs employs a first binding reagent that binds to CEA and a second binding reagent that binds to a tetraspanin.

77. The method of any claims 73-76, wherein the biological sample is plasma, blood, serum, or a liquid biopsy.

78. The method of any of claims 73-77, wherein the CEA+EVs are CEA+/CD73+EVs.

79. The method of any of claims 73-78, wherein performing an assay on a biological sample from the subject to detect CEA+EVs comprises performing a 3-marker proximity assay that employs a first binding reagent that binds CEA, and a second and third binding reagent that bind CD81 and CD73, respectively, wherein the first binding reagent is a capture reagent and the second and third binding reagents are proximity probes.

80. The method of any of claims 73-79, wherein the reference is an equivalent biological sample from a normal subject that does not have colorectal cancer.

81. The method of any of claims 73-80, comprising determining that the subject has stage III or stage IV CRC.

82. The method of any of claims 73-81, wherein the treatment regimen is selected from the group consisting of administration of a targeted drug, chemotherapy, immunotherapy, radiation therapy, surgery, or a combination of any of the above.

83. The method of any of claims 19-24, 38-43, 44-49, 50-62, 63, 64-72, 73-82, wherein if the level of biomarkers at the second timepoint is 1.2 fold, 1.5 fold, 2 fold, 3 fold, 4 fold, 5 fold, or 10 fold greater than the first, then the subject is not responding favorably to treatment.

84. The method of any of claims 19-24, 38-43, 44-49, 50-62, 63, 64-72, 73-82, wherein the level of biomarkers at the second timepoint is 1.2 fold, 1.5 fold, 2 fold, 3 fold, 4 fold, 5 fold, or 10 fold lower than the first, then the subject is responding favorably to treatment.

85. A system comprising:
one or more data processors; and
a non-transitory computer readable storage medium containing instructions which, when executed on the one or more data processors, cause the one or more data processors to perform part or all of the method of any one of claims 1 to 82.

86. A non-transitory computer readable medium, comprising instructions configured to cause one or more data processors to perform part or all of the method of any one of claims 1 to 84.

87. A non-transitory computer readable medium having stored thereon a computer program which, when executed by a computer system operably connected to an assay system configured to measure with a multiplexed binding assay, levels of a plurality of biomarkers comprising soluble CEA, soluble CD73, CEA+CD73+EV, CEA+EV, CD73+EV, or any combination thereof, in a sample from a human subject, causes the computer system to perform a method of predicting overall survival, progression free survival, duration of response, or a combination thereof in the subject receiving colorectal cancer treatment, the method comprising:
(a) fitting the measured levels of the plurality of biomarkers to a response surface model as a function of overall survival, progression free survival, duration of response, or a combination thereof;
(b) computing a cost function; and
(c) selecting an overall survival, progression free survival, duration of response that minimizes the cost function, thereby identifying the overall survival, progression free survival, duration of response, or combination thereof.

88. A method for managing treatment of a subject diagnosed with CRC, comprising:
receiving biomarker data corresponding to a biological sample obtained from the subject;
computing a response score that predicts a likelihood of responsiveness to continued treatment using quantification data identified from the biomarker data;
wherein the biomarker comprises soluble CEA, CEA+CD73+EV, CEA+EV, CD73+EV, soluble CD73 or any combination thereof; and
generating a treatment response output based on the response score.

89. The method of claim 88, wherein generating the treatment response output comprises:
generating a predicted response to a treatment based on whether the response score is above a selected threshold.

90. The method of claim 89, wherein the selected threshold is 1.0.
